(19) United States
(12) Patent Application Publication
(10) Pub. No.: US 2006/0293374 A1
(43) Pub. Date: Dec. 28, 2006

(54) Substituted isothiazolones

(76) Inventor: Scott A. Beers, Flemington, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/453,382

(22) Filed: Jun. 15, 2006

Related U.S. Application Data
(60) Provisional application No. 60/694,084, filed on Jun. 24, 2005.

(51) Int. Cl. A61K 31/425; C07D 417/02; C07D 275/02

(52) U.S. Cl. 514/372; 548/213

(57) ABSTRACT

This invention is directed to a compound of Formula (I): 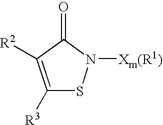
and pharmaceutically acceptable forms thereof useful as inhibitors of cPLA₂ and a method for preventing, treating or ameliorating a cPLA₂ mediated inflammatory related disease, disorder or condition using a compound of Formula (I) and, more particularly, for preventing, treating or ameliorating a cPLA₂ mediated inflammatory related disease, disorder or condition which results from the cellular secretion of TXB₂ or LTB₄.

### CROSS REFERENCE TO RELATED APPLICATIONS

This present application claims benefit of U.S. Provisional Patent Application Ser. No. 60/694084, filed Jun. 24, 2005, which is incorporated herein by reference in its entirety and for all purposes.

### BACKGROUND OF THE INVENTION

Cytosolic Phospholipase A₂ (cPLA₂) is an enzyme that catalyzes the hydrolysis of membrane phospholipids at the sn-2 position to release fatty acids and lysophospholipids. Because cPLA₂ is highly selective for phospholipids containing arachidonic acid (AA) at the sn-2 position, cPLA₂ is the principal enzyme responsible for intracellular generation of arachidonic acid.

Lysophospholipids are a second product resulting from phospholipid hydrolysis mediated by cPLA₂ and are metabolic precursors for platelet activating factor. Further evidence of the central role played by cPLA₂ in inflammation is provided by “knock out” mice, which are genetically deficient for the cPLA₂ gene.

In addition, the activation of cPLA2 and the subsequent increase in phospholipid hydrolysis has been shown to cause free fatty acids, diacylglycerols and lysophospholipids to accumulate and result in neuronal damage. cPLA₂ activity has been shown to be significantly elevated in many neurodegenerative disease states such as schizophrenia, multiple sclerosis, or as a result of head injury or stroke.

### SUMMARY OF THE INVENTION

The present invention provides compounds of Formula (I): 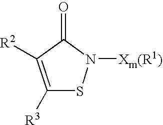
and pharmaceutically acceptable forms thereof as inhibitors of cPLA₂.

The present invention further provides compounds of Formula (I) as cPLA₂ inhibitors useful for preventing the cellular secretion of thromboxane B₂ (TXB₂) and leukotriene B₄ (LTB₄).

The present invention provides a method for preventing, treating or ameliorating a cPLA₂ mediated inflammatory related disease, disorder or condition using a compound of Formula (I) and, more particularly, for preventing, treating or ameliorating an inflammatory related disease, disorder or condition which results from the cellular secretion of TXB₂ or LTB₄.

The inflammatory related disease, disorder or condition contemplated in the method of the present invention includes, but is not limited to, rheumatoid arthritis, inflammatory bowel disease, septic shock, osteoporosis, osteoarthritis, neuropathic pain, psoriasis, allergic inflammation in the lung, chronic obstructive pulmonary disorders, multiple sclerosis, Alzheimer's disease, stroke, ischemia or schizophrenia.

### DETAILED DESCRIPTION OF THE INVENTION

The invention is a compound of Formula (I): 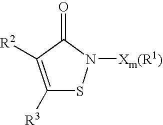
and pharmaceutically acceptable forms thereof, wherein:
X is —(CHR₄)—;
m is 0, 1, 2 or 3;
R¹ is -aryl-R₅, -heterocyclyl-R₆R₇ or —(C₃-C₁₄) cycloalkyl-R₆;
R² and R³ are each hydrogen or halogen;
R₄ is hydrogen, lower alkyl or R¹;
R₅ is hydrogen, halogen, heterocyclyl-R₇R_{8,} acyl, lower alkenyl-R_{9,} lower alkynyl-R_{9,} —SO₂—R_{10,} -carbonylaryl-R₉ or alkoxycarbonyl-;
R₆ is hydrogen, lower alkyl, acyl, halogen, aryl or heterocyclyl;
R₇ and R₈ are each hydrogen, lower alkyl or halogen;
R₉ is hydrogen, acyl or aryl, wherein aryl is optionally substituted by one or more nitrile or halogen;
R₁₀ is alkyl or aryl, wherein aryl is optionally substituted by one or more halogen;
wherein when XR¹ is —(CH₂)ₘ-(heterocyclyl)-R₆R₇, then heterocyclyl is other than pyrrolidinyl, 3(2H)-isothiazolone, piperidinyl, morpholinyl, benzisothiazol-3(2H)-one and 4H-1,3,2-benzoxazaphosphorin-4-one.

An example of the present invention is a compound of Formula (I) and pharmaceutically acceptable forms thereof, wherein X is —(CHR₄)—.

An example of the present invention is a compound of Formula (I) and pharmaceutically acceptable forms thereof, wherein m is 0 or 1.

An example of the present invention is a compound of Formula (I) and pharmaceutically acceptable forms thereof, wherein R¹ is -aryl-R₅, -heterocyclyl-R₆R₇ or —(C₃-C₁₄) cycloalkyl-R₆ and heterocyclyl is other than pyrrolidinyl, 3(2H)-isothiazolone, piperidinyl, morpholinyl, benzisothiazol-3(2H)-one and 4H-1,3,2-benzoxazaphosphorin-4-one.

An example of the present invention is a compound of Formula (I) and pharmaceutically acceptable forms thereof, wherein R² and R³ are each hydrogen or halogen.

An example of the present invention is a compound of Formula (I) and pharmaceutically acceptable forms thereof, wherein R₄ is hydrogen, lower alkyl or R¹.

An example of the present invention is a compound of Formula (I) and pharmaceutically acceptable forms thereof, wherein R₅ is hydrogen, heterocyclyl-R₇R₈, acyl, lower alkenyl-R₉, lower alkynyl-R₉, —SO₂—R₁₀, -carbonylaryl-R₉ or alkoxycarbonyl-.

An example of the present invention is a compound of Formula (I) and pharmaceutically acceptable forms thereof, wherein R₆ is hydrogen, lower alkyl, halogen, aryl or heterocyclyl.

An example of the present invention is a compound of Formula (I) and pharmaceutically acceptable forms thereof, wherein R₇ and R₈ are each hydrogen, lower alkyl or halogen.

An example of the present invention is a compound of Formula (I) and pharmaceutically acceptable forms thereof, wherein R₉ is hydrogen, acyl or aryl, wherein aryl is optionally substituted by one or more nitrile or halogen.

An example of the present invention is a compound of Formula (I) and pharmaceutically acceptable forms thereof, wherein R₁₀ is alkyl or aryl, wherein aryl is optionally substituted by one or more halogen.

The present invention further includes a method for preventing, treating or ameliorating a cPLA₂ mediated inflammatory related disease, disorder or condition in a patient in need thereof comprising administering to the patient an effective amount of a compound of Formula (I).

An example of the method of the present invention includes preventing, treating or ameliorating a cPLA₂ mediated inflammatory related disease, disorder or condition which results from the cellular secretion of TXB₂ or LTB₄ in a patient in need thereof comprising administering to the patient an effective amount of a compound of Formula (I).

An example of the method of the present invention includes preventing, treating or ameliorating a disease, disorder or condition including, but not limited to, rheumatoid arthritis, inflammatory bowel disease, septic shock, osteoporosis, osteoarthritis, neuropathic pain, psoriasis, allergic inflammation in the lung, chronic obstructive pulmonary disorders, multiple sclerosis, Alzheimer's disease, stroke, ischemia or schizophrenia.

An example of the compound of Formula (I) and pharmaceutically acceptable forms thereof is a compound wherein Xₘ(R¹), R² and R³ are dependently selected from:

| Cpd | Xₘ(R¹) | R² | R³ |
|---|---|---|---|
| 1 | CH₂-naphthalen-1-yl | H | Cl; |
| 2 | CH₂-naphthalen-1-yl | H | H; |
| 3 | CH₂-indan-1-yl | H | Cl; |
| 4 | CH₂-1,2,3,4-tetrahydro- | H | Cl; |
| | naphthalen-1-yl | | |
| 5 | CH(CH₃)-naphthalen-1-yl | H | Cl; |
| 6 | CH₂-3-Cl-benzo[b]thien-2-yl | H | Cl; |
| 7 | CH₂-benzo[b]thien-2-yl | H | Cl; |
| 8 | CH₂-3-Cl-benzo[b]thien-2-yl | H | H; |
| 9 | CH₂-benzo[b]thien-2-yl | H | H; |
| 10 | CH₂-benzo[b]thien-3-yl | Cl | Cl; |
| 11 | CH₂-benzo[b]thien-3-yl | H | Cl; |
| 12 | CH₂-benzo[b]thien-3-yl | H | H; |
| 13 | CH₂-naphthalen-2-yl | H | H; |
| 14 | CH₂-naphthalen-2-yl | H | Cl; |
| 15 | CH₂-naphthalen-2-yl | Cl | Cl; |
| 16 | CH₂-2,3-dihydro-benzofuran-5-yl | H | Cl; |
| 17 | CH₂-2,3-dihydro-benzofuran-5-yl | H | H; |
| 18 | CH₂-5-Cl-benzo[b]thien-3-yl | Cl | Cl; |
| 19 | CH₂-5-Cl-benzo[b]thien-3-yl | H | Cl; |
| 20 | CH₂-5-Cl-benzo[b]thien-3-yl | H | H; |
| 21 | CH₂-4-[1,2,3]thiadiazol-4-yl- | H | Cl; |
| | phenyl | | |
| 22 | CH₂-4-[1,2,3]thiadiazol-4-yl- | H | H; |
| | phenyl | | |
| 23 | CH₂-4-furan-2-yl-phenyl | H | Cl; |
| 24 | CH₂-4-[1,2,3]thiadiazol-4-yl- | Cl | Cl; |
| | phenyl | | |
| 25 | CH₂-4-C(O)CH₃-phenyl | H | H; |
| 26 | CH₂-4-(2-CH₃)-thiazol-4-yl- | H | H; |
| | phenyl | | |
| 27 | CH₂-4-CH═CH₂-phenyl | H | Cl; |
| 28 | CH₂-4-C≡C-(3-Cl)-phenyl-phenyl | Cl | Cl; |
| 29 | CH₂-4-SO₂CH₃-phenyl | Cl | Cl; |
| 30 | CH(CH₃)-4-SO₂CH₃-phenyl | Cl | Cl; |
| 31 | CH₂-4-(2-CH₃)-thiazol-4-yl- | H | Cl; |
| | phenyl | | |
| 32 | CH₂-4-[1,2,3]thiadiazol-4-yl- | H | Cl; |
| | phenyl | | |
| 33 | CH(phenyl-pyridin-2-yl) | Cl | Cl; |
| 34 | CH₂-4-C(O)-phenyl-phenyl | Cl | Cl; |
| 35 | CH₂-4-C(O)-phenyl-phenyl | H | Cl; |
| 36 | CH₂-4-SO₂-(4-F)-phenyl-phenyl | H | Cl; |
| 37 | CH₂-4-CO₂CH₃-phenyl | H | Cl; |
| 38 | CH₂-4-SO₂-phenyl-phenyl | H | Cl; |
| 39 | (1S,2R)-2-phenyl-cyclopropyl | Cl | Cl; |
| 40 | CH₂-4-C≡CH-phenyl | H | Cl; |
| 41 | CH₂-4-SO₂(CH₂)₄CH₃-phenyl | H | Cl; |
| 42 | (1S,2R)-2-phenyl-cyclopropyl | H | Cl; |
| 43 | CH₂-4-C≡C-(3-CN)-phenyl-phenyl | H | Cl; |
| 44 | 3-(5-Cl-2-CH₃)-thiazol-4-yl- | H | Cl; |
| | phenyl | | |
| 45 | 3-(5-Cl-2-CH₃)-thiazol-4-yl- | H | H; |
| | phenyl | | |
| 46 | 3-(2-CH₃)-thiazol-4-yl-phenyl | H | H; |
| 47 | 3-(2-CH₃)-thiazol-4-yl-phenyl | H | Cl; |
| 48 | CH₂-3-(2-CH₃)-thiazol-4-yl- | H | H; |
| | phenyl | | |
| 49 | CH₂-3-furan-2-yl-phenyl | H | H; |
| 50 | CH₂-3-pyridin-4-yl-phenyl | H | H; |
| 51 | CH₂-4-C(O)-phenyl-phenyl | H | H; |
| 52 | CH₂-5-Cl-2-phenyl-thiazol-4-yl | H | Cl; |
| 53 | CH₂-2-phenyl-thiazol-4-yl | H | Cl; |
| 54 | CH₂-2-phenyl-thiazol-4-yl | H | H; |
| 55 | CH₂-4-(4-Cl)-pyrazol-1-yl-phenyl | H | Cl; |
| 56 | CH₂-4-pyrazol-1-yl-phenyl | H | Cl; |
| 57 | CH₂-4-(4-Cl)-pyrazol-1-yl-phenyl | H | H; |
| 58 | CH₂-4-pyrazol-1-yl-phenyl | H | H; |
| 59 | CH₂-3-C(O)H-phenyl | H | Cl; |
| 60 | CH₂-3-C≡CC(O)H-phenyl | H | Cl; |
| 61 | CH₂-5-Cl-2-thien-2-yl-thiazol-4-yl | H | Cl; |
| 62 | CH₂-2-thien-2-yl-thiazol-4-yl | H | Cl; |
| 63 | CH₂-5-Cl-2-thien-2-yl-thiazol-4-yl | H | H; |
| 64 | CH₂-2-thien-2-yl-thiazol-4-yl | H | H; and |
| 65 | CH₂-4-C(O)CH₃-phenyl | Cl | Cl. |

An example of the compound of Formula (I) and a pharmaceutically acceptable form thereof is a compound selected from: 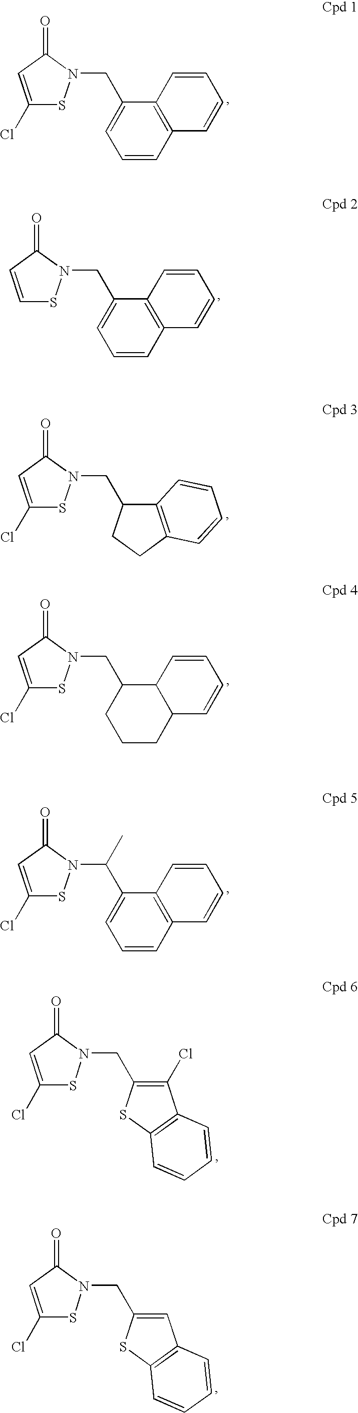 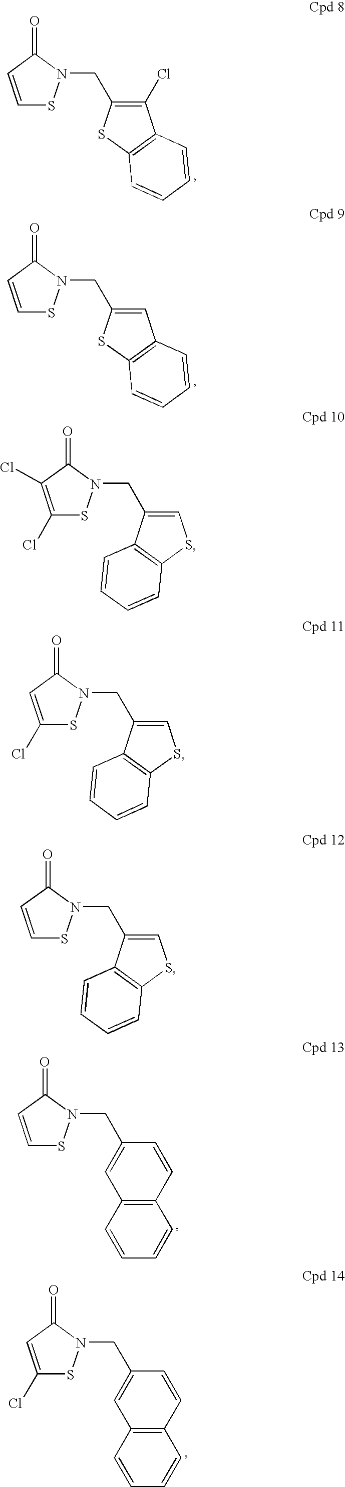 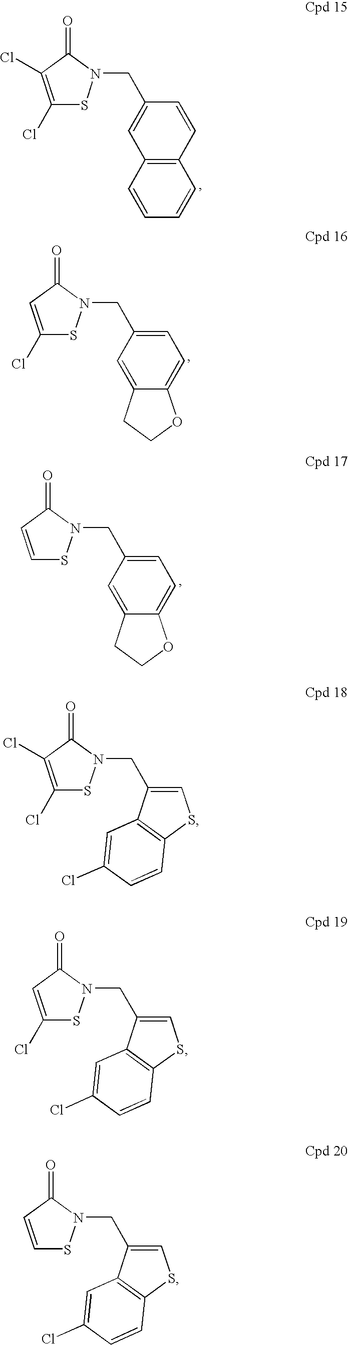 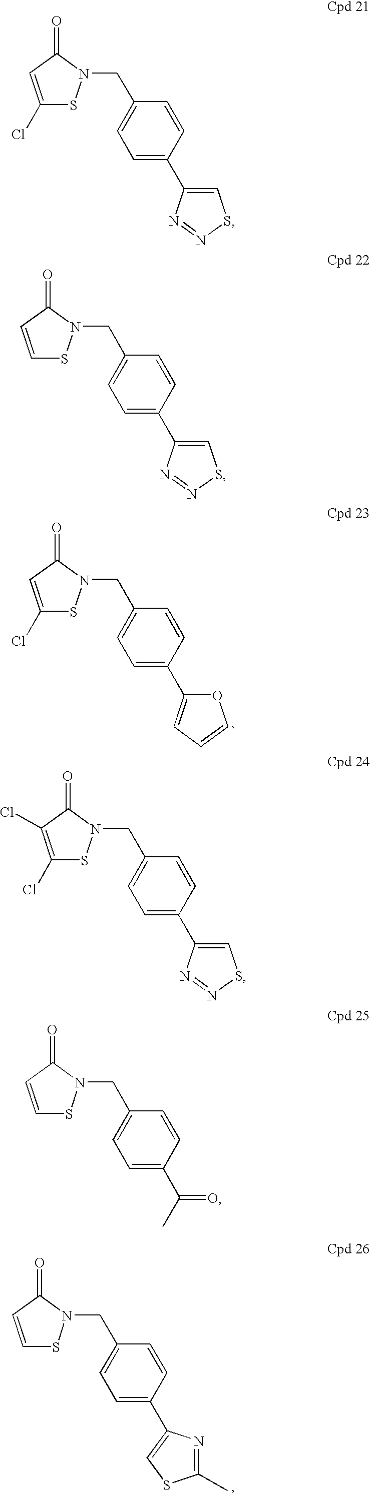 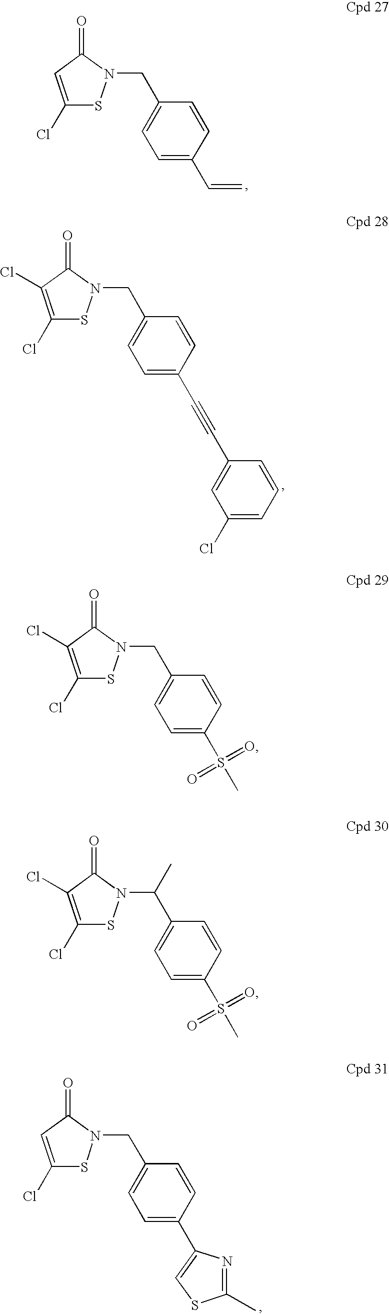 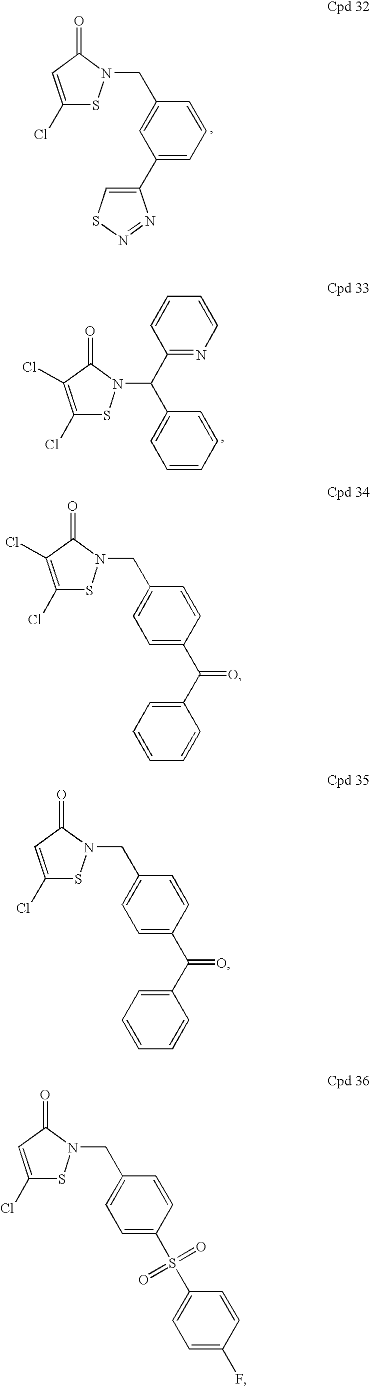 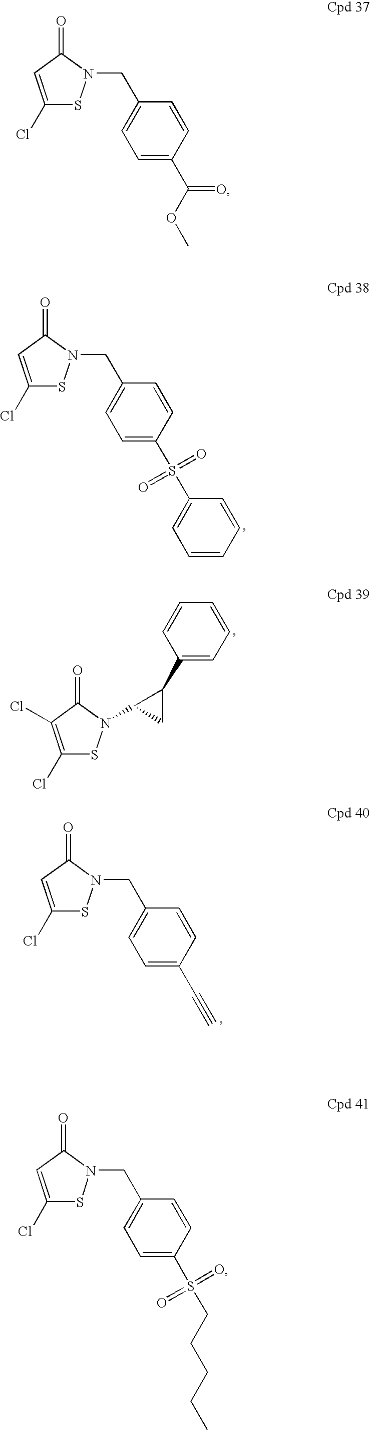 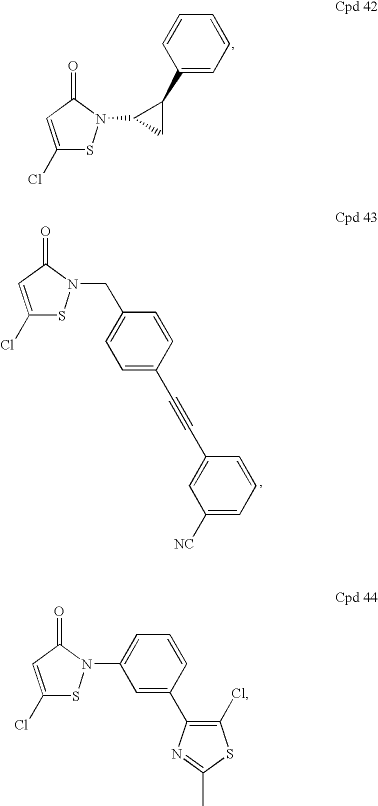 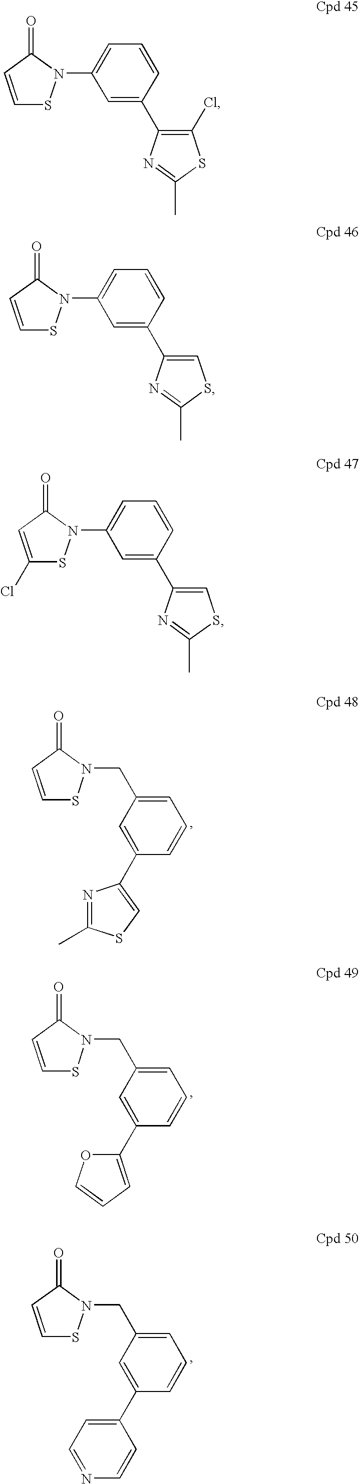 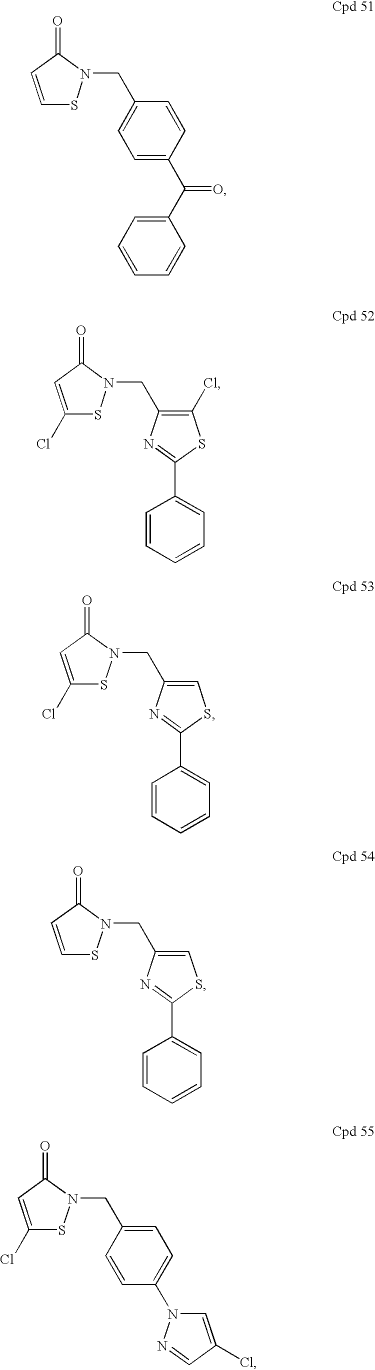 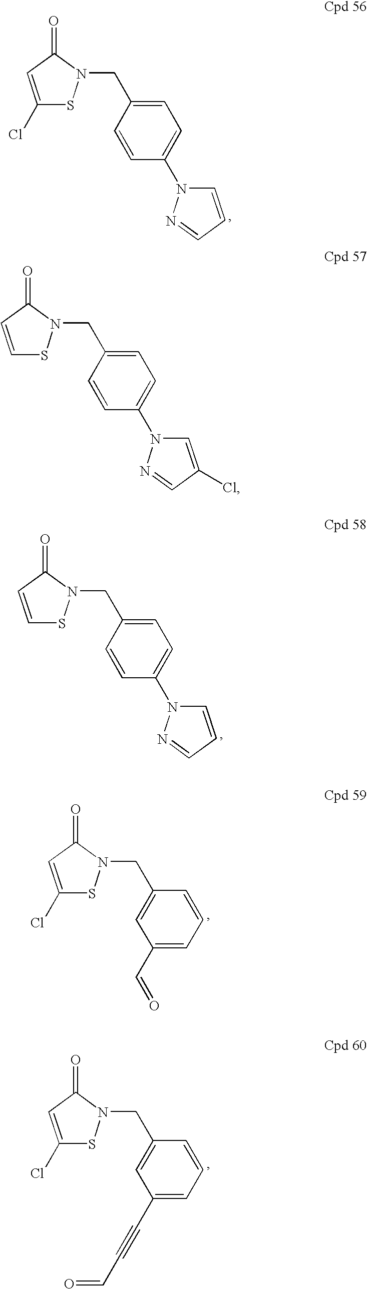 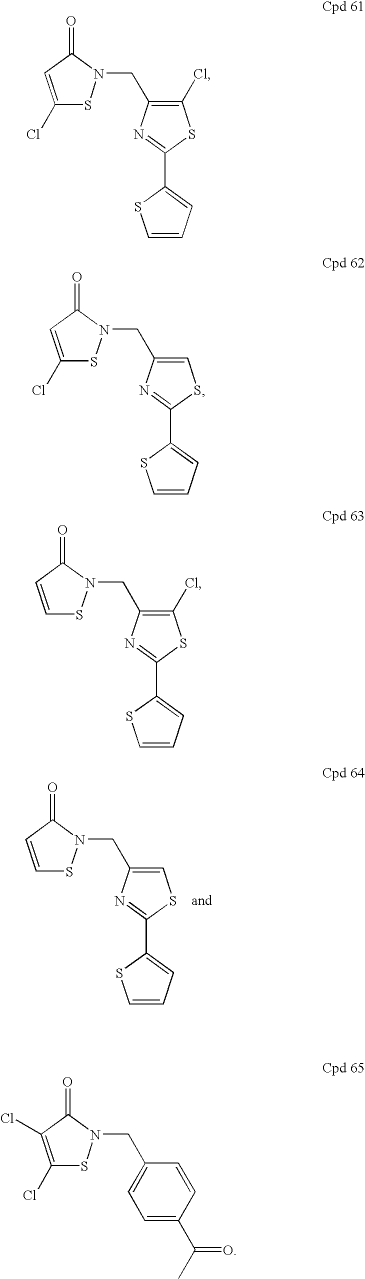
Chemical Definitions & Nomenclature

Bond lines drawn into a ring system from a substituent variable indicate that the substituent may be attached to any of the substitutable ring atoms.

The term “alkyl” means a saturated aliphatic branched or straight-chain monovalent hydrocarbon radical or linking group having a specified number of carbon atoms, wherein the alkyl radical is derived by the removal of one hydrogen atom from a carbon atom and the alkyl linking group is derived by the removal of one hydrogen atom from each of two carbon atoms in the chain.

The term “C₁₋₈alkyl” means a radical or linking group having from 1-8 carbon atoms in a linear or branched arrangement. “C₁₋₆alkyl” includes methyl, ethyl, propyl, butyl, pentyl or hexyl and the like. Alkyl radicals may be attached to a core molecule via a terminal carbon atom or via a carbon atom within the chain. Similarly, substituent variables may be attached to an alkyl linking group when allowed by available valences.

“Alkenyl” means an alkyl radical or linking group having at least two carbon atoms and at least one carbon-carbon double bond, wherein the double bond is derived by the removal of one hydrogen atom from each of two adjacent carbon atoms. The term “C₂₋₈alkenyl” means a radical or linking group having from 2-8 carbon atoms in a linear or branched arrangement and includes ethenyl or propenyl and the like.

“Alkynyl” means an alkyl radical or linking group having at least two carbon atoms and at least one carbon-carbon triple bond, wherein the triple bond is derived by the removal of two hydrogen atoms from each of two adjacent carbon atoms. The term “C₂₋₈alkynyl” means a radical or linking group having from 2-8 carbon atoms in a linear or branched arrangement and includes ethynyl or propynyl and the like.

“Alkoxy” means an alkyl radical or linking group attached through an oxygen linking atom. The term “C₁₋₄alkoxy” means a radical or linking group having from 1-4 carbon atoms in a linear or branched arrangement and includes methoxy, ethoxy, propoxy or butoxy and the like. An alkoxy radical may be attached to a core molecule and further substituted as a linking group where indicated.

“Cycloalkyl” means a monovalent saturated or partially unsaturated monocyclic, polycyclic or bridged ring system radical. Cycloalkyl ring systems include cyclopropyl, cyclobutyl, cyclopentyl, cyclohexyl, cycloheptyl, cyclooctyl, indanyl or 1,2,3,4-tetrahydro-naphthalenyl and the like.

“Aryl” means an monovalent unsaturated cycloalkyl radical. Aryl ring systems include phenyl, naphthalenyl, fluorenyl, indenyl, azulenyl or anthracenyl and the like.

“Hetero,” when used as a prefix for a ring system, refers to the replacement of at least one carbon atom member in the ring system with a heteroatom selected from N, O, S, S(O), or SO₂. A hetero ring may have 1, 2, 3, or 4 carbon atom members replaced by a nitrogen atom. Alternatively, a ring may have 0, 1, 2, or 3 nitrogen atom members and 1 oxygen or sulfur atom member. Alternatively, up to two adjacent ring members may be heteroatoms; wherein 1 heteroatom is nitrogen and the other heteroatom is selected from N, S, or O.

“Heterocyclyl” means a “hetero” ring system radical having an unsaturated, partially unsaturated or saturated cycloalkyl ring as the core molecule. Heterocyclyl ring systems include unsaturated ring systems such as furyl, thienyl, pyrrolyl, oxazolyl, thiazolyl, imidazolyl, pyrazolyl, isoxazolyl, isothiazolyl, oxadiazolyl, triazolyl, thiadiazolyl, pyridinyl, pyridazinyl, pyrimidinyl, pyrazinyl, indolizinyl, indolyl, azaindolyl, isoindolyl, benzo[b]furyl, benzo[b]thienyl, indazolyl, azaindazolyl, benzimidazolyl, benzthiazolyl, benzoxazolyl, benzisoxazolyl, benzothiadiazolyl, benzotriazolyl, purinyl, 4H-quinolizinyl, quinolinyl, isoquinolinyl, cinnolinyl, phthalzinyl, quinazolinyl, quinoxalinyl, 1,8-naphthyridinyl, pteridinyl and the like and partially unsaturated or saturated ring systems such as pyrrolinyl, pyrrolidinyl, 1,3-dioxolanyl, imidazolinyl (also referred to as 4,5-dihydro-1H-imidazolyl), imidazolidinyl, pyrazolinyl, pyrazolidinyl, tetrazolyl, tetrazolidinyl, piperidinyl, 1,4-dioxanyl, morpholinyl, 1,4-dithianyl, thiomorpholinyl, piperazinyl, azetidinyl, azepanyl, hexahydro-1,4-diazepinyl, hexahydro-1,4-oxazepanyl, tetrahydro-furanyl, tetrahydro-thienyl, tetrahydro-pyranyl, tetrahydro-pyridazinyl, 1,3-benzodioxolyl, 2,3-dihydro-1,4-benzodioxinyl, 2,3-dihydro-benzofuranyl and the like.

“Substituted” means the independent replacement of one or more hydrogen atoms within a radical with that amount of substituents allowed by available valences.

“Dependently selected” means that one or more substituent variables are specified in an indicated combination.

In general, IUPAC nomenclature rules are used herein.

Pharmaceutically Acceptable Forms

Certain compounds of Formula (I) may exist in various stereoisomeric or tautomeric forms. The invention encompasses all such compounds, including active compounds in the form of essentially pure enantiomers, racemic mixtures and tautomers.

The compounds of the invention may be present in the form of pharmaceutically acceptable salts. For use in medicines, the salts of the compounds of this invention refer to non-toxic “pharmaceutically acceptable salts.” FDA-approved pharmaceutically acceptable salt forms include pharmaceutically acceptable acidic/anionic or basic/cationic salts.

Pharmaceutically acceptable acidic/anionic salts include the acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, methylbromide, methylnitrate, methylsulfate, mucate, napsylate, nitrate, pamoate, pantothenate, phosphate/diphosphate, polygalacturonate, salicylate, stearate, subacetate, succinate, sulfate, tannate, tartrate, teoclate, tosylate and triethiodide salts.

The compounds of the invention include pharmaceutically acceptable anionic salt forms, wherein the anionic salts include the acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, methylbromide, methylnitrate, methylsulfate, mucate, napsylate, nitrate, pamoate, pantothenate, phosphate/diphosphate, polygalacturonate, salicylate, stearate, subacetate, succinate, sulfate, tannate, tartrate, teoclate, tosylate and triethiodide salts.

The anionic salt form of a compound of the invention includes an anionic salt selected from the acetate, bromide, camsylate, chloride, edisylate, fumarate, hydrobromide, hydrochloride, iodide, isethionate, lactate, mesylate, napsylate, salicylate, sulfate and tosylate salts.

During any of the processes for preparation of the compounds of the invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, 3^{rd} Edition, John Wiley & Sons, 1999. The protecting groups may be removed at a convenient subsequent stage using methods known in the art.

The invention includes compounds of various isomers and mixtures thereof. The term “isomer” refers to compounds that have the same composition and molecular weight but differ in physical and/or chemical properties. Such substances have the same number and kind of atoms but differ in structure. The structural difference may be in constitution (geometric isomers) or in an ability to rotate the plane of polarized light (stereoisomers).

The term “stereoisomer” means isomers of identical constitution that differ in the spatial arrangement of their atoms. Enantiomers and diastereomers are stereoisomers wherein an asymmetrically substituted carbon atom acts as a chiral center. The term “chiral” means a molecule that is not superimposable on its mirror image, implying the absence of an axis and a plane or center of symmetry. The term “enantiomer” means one of a pair of molecular species that are mirror images of each other and are not superimposable. The term “diastereomer” means stereoisomers that are not related as mirror images. The symbols “R” and “S” represent the configuration of substituents around a chiral carbon atom(s).

The term “racemate” or “racemic mixture” means a compound of equimolar quantities of two enantiomeric species, wherein the compound is devoid of optical activity. The term “optical activity” means the degree to which a chiral molecule or non-racemic mixture of chiral molecules rotates the plane of polarized light.

“Geometric isomer” means isomers that differ in the orientation of substituent atoms in relationship to a carbon-carbon double bond, to a cycloalkyl ring, or to a bridged bicyclic system. Substituent atoms (other than H) on each side of a carbon-carbon double bond may be in an E or Z configuration. In the “E” configuration, the substituents are on opposite sides in relationship to the carbon-carbon double bond. In the “Z” configuration, the substituents are oriented on the same side in relationship to the carbon-carbon double bond.

The isomeric descriptors (“R,” “S,” “E,” and “Z”) indicate atom configurations relative to a core molecule and are intended to be used as defined in the literature.

The compounds of the invention may be prepared as individual isomers by either isomer-specific synthesis or resolved from an isomeric mixture. Conventional resolution techniques include combining the free base (or free acid) of each isomer of an isomeric pair using an optically active acid (or base) to form an optically active salt (followed by fractional crystallization and regeneration of the free base), forming an ester or amide of each of the isomers of an isomeric pair by reaction with an appropriate chiral auxiliary (followed by fractional crystallization or chromatographic separation and removal of the chiral auxiliary), or separating an isomeric mixture of either an intermediate or a final product using various well known chromatographic methods.

Furthermore, compounds of the invention may have one or more polymorph or amorphous crystalline forms. Said forms are included in the scope of the invention. In addition, some of the compounds may form solvates with water (i.e., hydrates) or common organic solvents. Said solvates are encompassed within the scope of this invention.

Therapeutic of Use

A compound of Formula (I) for use in the present invention includes a compound having a mean inhibition binding constant (IC₅₀) of between about 5 μM to about 0.01 nM; between about 1 μM to about 0.01 nM; between about 800 nM to about 0.01 nM; between about 200 nM to about 0.01 nM; between about 100 nM to about 0.01 nM; between about 80 nM to about 0.01 nM; between about 20 nM to about 0.01 nM; between about 10 nM to about 0.1 nM; or about 1 nM.

The present invention includes a method for preventing, treating or ameliorating a cPLA₂ mediated inflammatory related disease, disorder or condition in a patient in need thereof comprising the step of administering to the patient an effective amount of a compound of Formula (I).

The present invention also includes a method for preventing, treating or ameliorating a cPLA₂ mediated inflammatory related disease, disorder or condition which results from the cellular secretion of TXB₂ or LTB₄ in a patient in need thereof comprising administering to the patient an effective amount of a compound of Formula (I).

An example of the present invention in the method for preventing, treating or ameliorating a cPLA₂ mediated inflammatory related disease, disorder or condition includes, and is not limited to, rheumatoid arthritis, inflammatory bowel disease, septic shock, osteoporosis, osteoarthritis, neuropathic pain, psoriasis, allergic inflammation in the lung, chronic obstructive pulmonary disorder, multiple sclerosis, Alzheimer's disease, stroke, ischemia or schizophrenia.

“Patient” means a mammal, preferably a human, who has a cPLA₂ mediated inflammatory related disease, disorder or condition or a cPLA₂ mediated inflammatory related disease, disorder or condition, which results from the cellular secretion of TXB₂ or LTB₄. Since both human and veterinary use are included within the scope of the invention, the method of the present invention further includes administering an effective amount of a compound of Formula (I) or a composition or medicament thereof for either human or veterinary use.

“Administering,” with respect to the methods of the invention, means a method for preventing, treating or ameliorating a disease, disorder or condition as described herein with a compound of the invention or prodrug, metabolite, or composition thereof. Such methods include administering an effective amount thereof at different times during the course of a therapy or concurrently in a combination form. The methods of the invention are to be understood as embracing all known therapeutic treatment regimens.

“Prodrug” means a pharmaceutically acceptable form of a functional derivative of a compound of the invention (or a salt thereof), wherein the prodrug may be: 1) a relatively active precursor which converts in vivo to an active prodrug component; 2) a relatively inactive precursor which converts in vivo to an active prodrug component; or 3) a relatively less active component of the compound that contributes to therapeutic biological activity after becoming available in vivo (i.e., as a metabolite). Conventional procedures for the selection and preparation of suitable prodrug derivatives are described, for example, in “Design of Prodrugs”, ed. H. Bundgaard, Elsevier, 1985.

“Metabolite” means a pharmaceutically acceptable form of a metabolic derivative of a compound of the invention(or a salt thereof), wherein the derivative is a relatively less active component of the compound that contributes to therapeutic biological activity after becoming available in vivo.

“Effective amount” means that amount of a compound of Formula (I) or pharmaceutical composition thereof that is effective in a patient to treat or ameliorate the symptoms of the disease, disorder or condition being treated. The effective amount of the compound is from about 0.001 mg/kg/day to about 300 mg/kg/day.

“Inflammatory related disease, disorder or condition” means a biological response such that there is discomfort or decreased life expectancy to the patient.

The present invention further includes a method for use of one or more compounds of Formula (I) in a combination product for preventing, treating or ameliorating an inflammatory related disease, disorder or condition in a patient in need thereof comprising administering to the patient an effective amount of the combination product.

“Combination product” means a pharmaceutical composition comprising one or more compounds of Formula (I) in combination with one or more therapeutic agents. The dosages of the compound of Formula (I) and the one or more therapeutic agents are adjusted when combined to achieve an effective amount. As those skilled in the art will appreciate, the effective amounts of the components comprising the combination product may be independently optimized and combined to achieve a synergistic result whereby the pathology is reduced more than it would be if the components of the combination product were used alone.

“Effective amount,” with respect to the methods of the invention further comprising a method for use of a combination product for preventing, treating or ameliorating an inflammatory related disease, disorder or condition also means that amount of the combination of one or more compounds of Formula (I) and one or more agents taken together so that the combined effect treats or ameliorates the symptoms of the disease, disorder or condition.

“Administering,” with respect to the methods of the invention further comprising a method for use of a combination product for preventing, treating or ameliorating the inflammatory related disease, disorder or condition, means administering an effective amount thereof by co-administration of the compound and the agent, sequential administration of the compound and the agent, administration of a single composition containing of the compound and the agent or simultaneous administration of separate, divided compositions containing the compound and the agent. The methods of the invention are therefore further to be understood as embracing all such regimes of simultaneous or alternating treatment regimens.

Therapeutic agents contemplated for use in a combination product of the invention include anti-inflammatory analgesic agents such as, but not limited to, a NSAID, a nitric oxide (NO) inhibitor, a cyclooxygenase (COX) inhibitor, a prostaglandin (PG) inhibitor, an interleukin inhibitor, a leukotriene (LT) inhibitor, an inflammatory or growth proliferation kinase inhibitor, an inflammatory cytokine inhibitor, a corticosteroid or an apoptosis inhibitor and the like.

Examples of a NSAID include analogs of carboxylic acid, acetic acid, propionic acid, salicylic acid, hydrotropic acid (such as suprofen), hydroxamic acid (such as tepoxalin), benzoic/xylylanthranilic acid (such as mefenamic acid), benzene acetic acid (such as ibuprofen, diclofenac or alclofenac), indole acetic acid (such as indomethacin), indene acetic acid (such as sulindac), toluoylpyrrole acetic acid (such as tolmetin), naphthalene acetic acid (such as naproxen), benzopyranopyridine acetic acid (such as pranoprofen), pyrazolidinedione (such as phenylbutazone or oxyphenbutazone) or benzthiazine (such as piroxicam) and the like or a salt or ester form or mixture thereof.

Embodiments of the present invention further include at least one NSAID wherein the NSAID is a cyclooxygenase, leukotriene or prostaglandin inhibitor, wherein the cyclooxygenase inhibitor is selected from a SLO-1, COX-1 or COX-2 inhibitor and the like, wherein the leukotriene inhibitor is a LTB₄ inhibitor and the like and wherein the prostaglandin inhibitor is a PGE₂ inhibitor and the like.

Embodiments of the invention include at least one NSAID wherein the NSAID is a 5LO-1, COX-1, COX-2, LTB₄ and TXB₂ inhibitor.

An embodiment of the invention includes an NSAID wherein the NSAID is a 5LO-1 inhibitor having an IC₅₀ of from about 0.010 μM to about 10 μM.

An embodiment of the invention includes an NSAID wherein the NSAID is a COX-1 inhibitor having an IC₅₀ of from about 0.06 μM to about 12 μM.

An embodiment of the invention includes an NSAID wherein the NSAID is a COX-2 inhibitor having an IC₅₀ of from about 0.05 μM to about 6 μM.

An embodiment of the invention includes an NSAID wherein the NSAID is a LTB₄ inhibitor having an IC₅₀ of from about 0.035 μM to about 2 μM.

An embodiment of the invention includes an NSAID wherein the NSAID is a TXB₂ inhibitor having an IC₅₀ of from about 0.002 μM to about 0.04 μM.

Certain embodiments of the invention further include at least one NSAID wherein the NSAID is selected from suprofen, tepoxalin or tolmetin.

Embodiments of the present invention include an inflammatory kinase inhibitor such as a MAP kinase inhibitor, wherein the MAP kinase inhibitor is a p38, ERK or JNK2 kinase inhibitor.

Embodiments of the present invention include a growth proliferation kinase inhibitor such as sirolimus (rapamycin).

Embodiments of the present invention include an inflammatory cytokine inhibitor such as a TNF-α inhibitor, an IL-1α inhibitor, IL-1β inhibitor, IL-6 inhibitor, IL-10 inhibitor, IL-12 inhibitor or an IL-17 inhibitor.

Pharmaceutical Compositions

A pharmaceutical composition of the present invention may, alternatively or in addition to a compound of Formula (I) comprise a pharmaceutically acceptable salt of a compound of Formula (I) or a prodrug or pharmaceutically active metabolite of such a compound or salt in admixture with a pharmaceutically acceptable carrier.

“Composition” means a product comprising at least a compound of the invention and a pharmaceutically acceptable carrier or any such alternatives to a compound of the invention and a pharmaceutically acceptable carrier, as well as any product that results, directly or indirectly, from such combinations. The invention further comprises mixing one or more of the compounds of the invention and a pharmaceutically acceptable carrier; and, includes those compositions resulting from such a process. Contemplated processes include both traditional and modern pharmaceutical techniques.

“Pharmaceutically acceptable carrier” means molecular entities and compositions that are of sufficient purity and quality for use in the formulation of a composition of the invention and that, when appropriately administered to a patient, do not produce an adverse, allergic, or other untoward reaction. Accordingly, a pharmaceutically acceptable formulation includes a composition or medicament for either human or veterinary use.

The composition may take a wide variety of forms to effectuate mode of administration including ocular, oral, sublingual, nasal (inhaled or insufflated), transdermal, rectal, vaginal, topical (with or without occlusion), intravenous (both bolus and infusion) and injection (intraperitoneally, subcutaneously, intramuscularly, intratumorally, or parenterally). Compounds may also be administrated directly to the nervous system including, but not limited to the intracerebral, intraventricular, intracerebroventricular, intrathecal, intracisternal, intraspinal and/or peri-spinal routes of administration by delivery via intracranial or intravertebral needles and/or catheters with or without pump devices.

The composition may be in a dosage unit such as a tablet, pill, capsule, syrup, elixir, emulsion, powder, granule, liposome, ion exchange resin, sterile ocular solution, or ocular delivery device (such as a contact lens and the like facilitating immediate release, timed release, or sustained release), solution or suspension (for parenteral or oral use), metered aerosol or liquid spray, drop, ampoule, auto-injector device, or suppository; for administration ocularly, orally, intranasally, sublingually, parenterally, or rectally or by inhalation or insufflation.

Compositions suitable for oral administration include solid forms such as pills, tablets, caplets, capsules (each including immediate release, timed release and sustained release formulations), granules and powders; and, liquid forms such as solutions, syrups, elixirs, emulsions and suspensions. Forms useful for ocular administration include sterile solutions or ocular delivery devices. Forms useful for parenteral administration include sterile solutions, emulsions and suspensions.

Alternatively, the composition may be administered in a form suitable for once-weekly or once-monthly administration. For example, an insoluble salt of the active compound may be adapted to provide a depot preparation for intramuscular injection (e.g., a decanoate salt) or to provide a solution for ophthalmic administration.

The dosage form containing the composition thereof contains an effective amount of a compound of Formula (I). The composition may contain from about 0.001 mg to about 5000 mg (preferably, from about 0.001 to about 500 mg) of a compound of the invention or salt form thereof and may be constituted into any form suitable for the mode of administration selected for a subject in need.

A contemplated effective amount may range from about 0.001 mg to about 300 mg/kg of body weight per day. Preferably, the range is from about 0.003 to about 100 mg/kg of body weight per day. Most preferably, the range is from about 0.005 to about 15 mg/kg of body weight per day. The composition may be administered according to a dosage regimen of from about 1 to about 5 times per day.

For oral administration, the composition is preferably in the form of a tablet or capsule containing, e.g., 0.01, 0.05, 0.1, 0.5, 1.0, 2.5, 5.0, 10.0, 15.0, 25.0, 50.0, 100, 150, 200, 250, or 500 milligrams of the compound of the invention for the symptomatic adjustment of the dosage to the patient to be treated. Optimal dosages will vary depending on factors associated with the particular patient being treated (e.g., age, weight, diet, time of administration, the severity and advancement of the condition being treated, the compound being employed, the mode of administration and the strength of the preparation and concomitant diseases). The use of either daily administration or post-periodic dosing may be employed. An optimal dose to be administered may be readily determined by those skilled in the art.

The oral composition is preferably formulated as a homogeneous composition wherein the compound of the invention is dispersed evenly throughout the mixture, which may be readily subdivided into dosage units containing equal amounts of a compound of the invention. Preferably, the compositions are prepared by mixing a compound of the invention (or pharmaceutically acceptable salt thereof) with one or more pharmaceutical carriers.

Because of their ease of administration, tablets and capsules represent an advantageous oral dosage unit form wherein solid pharmaceutical carriers are employed. If desired, tablets may be sugarcoated or filmcoated using standard techniques. Tablets may also be coated or otherwise compounded to provide a prolonged, control-release therapeutic effect.

Compounds of the invention may also be administered via a slow release composition, wherein the composition includes a biodegradable slow release carrier (e.g., a polymeric carrier) or a pharmaceutically acceptable non-biodegradable slow release carrier (e.g., an ion exchange carrier).

Biodegradable and non-biodegradable slow release carriers are well known in the art. Biodegradable carriers are used to form particles or matrices which retain an active agent(s) and which slowly degrade/dissolve in a suitable environment (e.g., aqueous, acidic, basic and the like) to release the agent.

The compound of Formula (I) may be incorporated for administration orally or by injection in a liquid form. The compounds may alternatively be administered parenterally via injection.

Compounds of the invention may be administered intranasally using a suitable intranasal vehicle. Compounds of the invention may be administered topically using a suitable topical transdermal vehicle or a transdermal patch. Administration via a transdermal delivery system requires a continuous rather than intermittent dosage regimen.

Synthetic Methods

Representative compounds of the invention can be synthesized in accordance with the general synthetic schemes described below and are illustrated more particularly in the specific examples that follow. The general schemes and specific examples are offered by way of illustration; the invention should not be construed as being limited by the chemical reactions and conditions expressed. The methods for preparing the various starting materials used in the schemes and examples are well within the skill of persons versed in the art.

The following abbreviations have the indicated meanings:

| | |
|---|---|
| Cpd | compound |
| DCM | dichloromethane |
| DMF | N,N-dimethylformamide |
| EtOAc | ethyl acetate |
| LAH | lithium aluminum hydride |
| MS | mass spectrum, refers to data shown as m/z (M + H)⁺ |
| min/hr(s) | minute/hour(s) |
| M.P. | melting point |
| RT/rt/r.t. | room temperature |
| TEA | triethylamine |
| TFA | trifluoroacetic acid |
| THF | tetrahydrofuran |

All commercially available chemicals were obtained from commercial suppliers and used without further purification. Particular components or equipment used in the examples, such as reaction vessels and the like, are also commercially available.

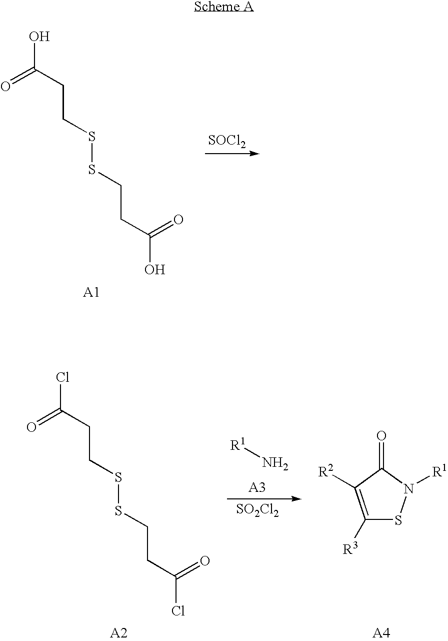

Scheme A shows the synthetic preparation of the isothiazolone ring system, which is used throughout the present invention. The methodology is an adaptation of that reported by Lewis et al., Isothiazoles I: 4-Isothiazolin-3-ones. A general Synthesis from 3,3′-Dithiodipropionamides J. Heterocyclic Chem. 1971, 8, 571-580.

A commercially available 3,3′-dithiodipropionic acid Compound A1 is treated with a catalytic amount of pyridine and an excess of thionyl chloride. The reaction is stirred under an inert atmosphere such as nitrogen for a period of from about 3 hrs to about 3 days. Evaporation of the excess thionyl chloride in vacuo gives the bis acid chloride Compound A2 as an oil which is used without further purification. The bis acid chloride is then reacted with an amine Compound A3 and TEA in DCM. After 3-18 hrs, sulfuryl chloride (3-5 equivalents) is added and the reaction is then stirred for an additional 3-18 h before being worked up and purified.

Embodiments of the compounds of the present invention include additional substitution on the R¹ substituent which may be accomplished using the procedures provided in the specific synthetic examples included herein as well as by using techniques, starting materials, reagents and conditions known to those skilled in the art.

Embodiments of the compounds of the present invention further include substitution on the R² and R³ substituent position which may be accomplished by addition of 3 equivalents of sulfuryl chloride to favor formation of the nonchlorinated isothiazolone ring (R² and R³ are hydrogen) or by addition of 4-5 equivalents to favor formation of mono and dihalogenated isothiazolones (R² and R³ are chlorine).

### EXAMPLE 1

### 4,5-dichloro-2-(4-iodobenzyl)-isothiazol-3-one (Intermediate Cpd 1d)

### 5-chloro-2-(4-iodobenzyl)-isothiazol-3-one (Intermediate Cpd 1e)

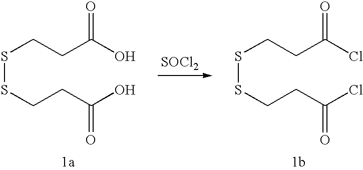

3-(2-carboxy-ethyldisulfanyl)-propionic acid Compound 1a (2.48 grams, 0.012 mol) was placed in a 100 mL round bottom flask. Pyridine (2 drops) and thionyl chloride (20 mL) were added and the mixture was stirred under an atmosphere of nitrogen for 16 hrs. The resulting clear solution was evaporated in vacuo and the residue oil was dissolved in DCM (30 mL) and again evaporated in vacuo to provide 3-(2-chlorocarbonyl-ethyldisulfanyl)-propionyl chloride Compound 1b.

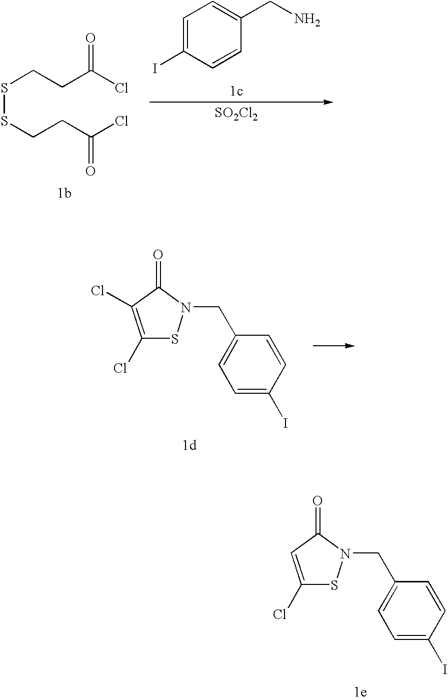

Compound 1b was then transferred via pipette to a second flask (cooled in an ice water bath) containing DCM (250 mL), 4-iodobenzylamine Compound 1c (5.50 grams, 0.024 mol) and TEA (3.3 mL, 0.024 mol). The reaction mixture was stirred for 3 hrs, then approximately 2 molar equivalents of sulfuryl chloride (4.00 mL, 0.050 mol) was added and the mixture was stirred at ambient temperature for an additional 3 hrs. The mixture was evaporated in vacuo and the resulting crude oil was dissolved in DCM (250 mL), washed with water, dried over magnesium sulfate and concentrated in vacuo. The crude product was chromatographed on a silica gel flash column using a mobile phase of hexane:EtOAc in a 2:1 ratio to afford the intermediate 4,5-dichloro-2-(4-iodo-benzyl)-isothiazol-3-one Compound 1d as a solid. M.P. 104.5-107° C.; ¹H NMR (CDCl₃) δ 7.71 (m, 2H), 7.07 (d, 2H), 4.89 (s, 2H).

Further elution of the same column produced the intermediate 5-chloro-2-(4-iodo-benzyl)-isothiazol-3-one Compound 1e. ¹H NMR (CDCl₃) δ 7.70 (d, 2H), 7.05 (d, 2H), 6.30 (s, 1H), 4.82 (s, 2H).

### EXAMPLE 2

### 2-(4-acetylbenzyl)-isothiazol-3-one (Compound 25)

### 2-[4-(2-methyl-thiazol-4-yl)-benzyl]-isothiazol-3-one (Compound 26)

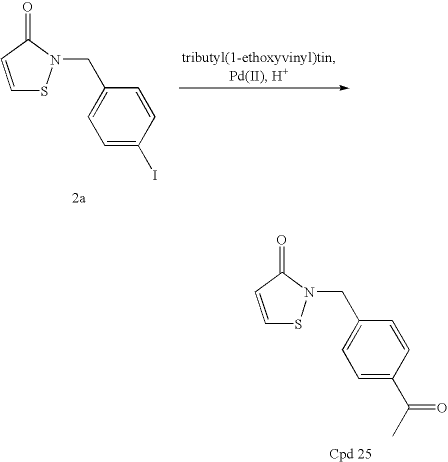

Using the procedure of Example 1 and 3 molar equivalents of sulfuryl chloride, a crude product was obtained and purified using silica gel flash chromatography eluted with EtOAc to provide intermediate 2-(4-iodo-benzyl)-isothiazol-3-one Compound 2a as a white solid. M.P. 83-84° C.; ¹H NMR (CDCl₃) δ 8.07 (d, 1H), 7.69 (d, 2H), 7.06 (d, 2H), 6.29 (d, 1H), 4.89 (s, 2H).

Intermediate Compound 2a (4.51 grams, 0.014 mol) was dissolved in dioxane (100 mL). Tributyl-(1-ethoxyvinyl)-tin (7.1 mL, 0.021 mol) and dichloro-bis(triphenylphosphine)-palladium (II) (1.00 grams, 10 mol %) were added and the reaction mixture was stirred at 115° C. for 30 min. The mixture was filtered, then water (10 mL) and concentrated hydrochloric acid (3 drops) were added to the filtrate. The mixture was stirred for an additional 40 min, then diluted with EtOAc (100 mL) and washed with water (3×100 mL). The organic layer was concentrated in vacuo and applied to a silica gel flash column and eluted with EtOAc to give Compound 25 (1.44 grams, 44%) as a white solid. ¹H NMR (CDCl₃) δ 8.11 (d, 1H), 7.96 (d, 2H), 7.40 (d, 2H), 6.32 (d, 1H), 5.00 (s, 2h), 2.60 (s, 3H).

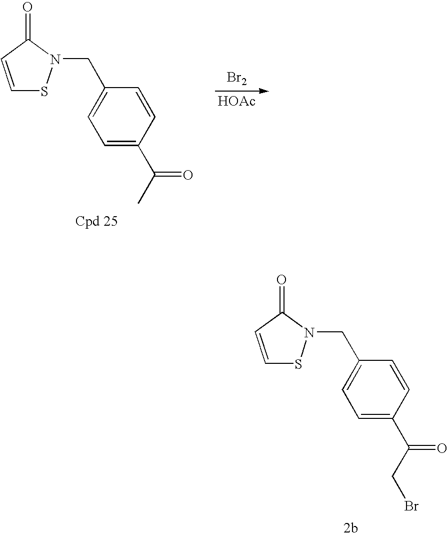

Compound 25 (2.19 grams, 9.40 mmol) was dissolved in acetic acid (30 mL). Bromine (0.50 mL) in acetic acid (10 mL) was added drop wise and the reaction mixture was stirred for 23 hrs. DCM (50 mL) was added and the mixture was washed with water (2×50 mL) then with saturated aqueous sodium bicarbonate. The organic layer was dried (magnesium sulfate), concentrated in vacuo and column chromatographed (silica gel) using a mobile phase of EtOAc to give 2-[4-(2-bromoacetyl)benzyl]-isothiazol-3-one Compound 2b (1.57 grams, 54%) as a red foam. ¹H NMR (CDCl₃) δ 8.13 (d, 1H), 7.99 (d, 2H), 7.42 (d, 2H), 6.33 (d, 1H), 5.01 (s, 2H), 4.45 (s, 2H).

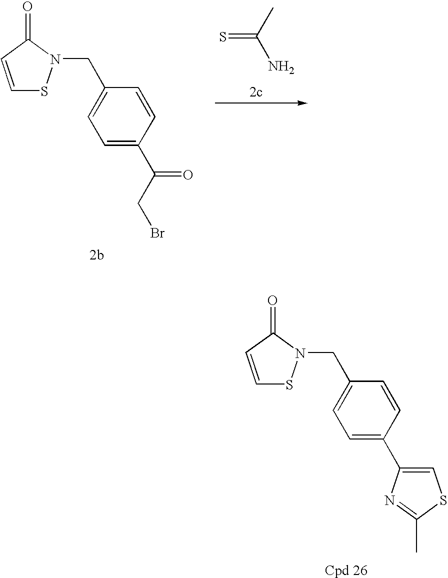

Intermediate Compound 2b (1.57 grams, 5.03 mmol) was dissolved in ethanol (50 mL) and thioacetamide Compound 2c (0.38 grams, 5.06 mmol) and pyridine (0.41 mL, 5.07 mmol) were added. The reaction mixture was stirred at reflux for 90 min, then poured into water and the product was extracted with DCM. The organic layer was dried (magnesium sulfate), concentrated in vacuo and chromatographed on a silica gel flash column eluted with EtOAc to give Compound 26 as a white solid. M.P. 150.5-152° C.; MS 289 (M+H⁺); ¹H NMR (CDCl₃) δ 8.03 (d, 1H), 7.86 (d, 2H), 7.35 (d, 2H), 7.32 (s, 1H), 6.30 (d, 1H), 4.96 (s, 2H), 2.77 (s, 3H).

### EXAMPLE 3

### 2-(3-furan-2-yl-benzyl)-isothiazol-3-one (Cpd 49)

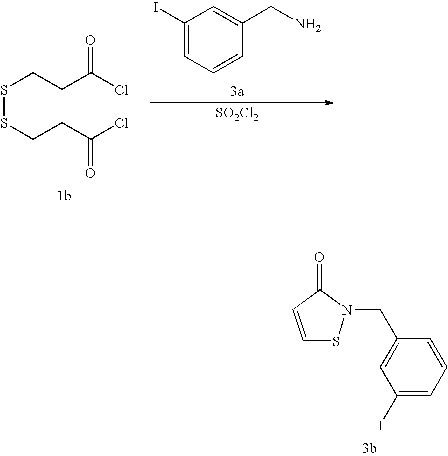

Using the procedure of Example 1 and 3-iodobenzylamine Compound 3a in place of Compound 1c and 3 molar equivalents of sulfuryl chloride, intermediate 2-(3-iodobenzyl)-isothiazol-3-one Compound 3b was obtained as a white solid. M.P. 54-54.5° C.; ¹H NMR (CDCl₃) δ 8.08 (d, 1H), 7.10 (t, 1H), 6.31 (d, 1H), 4.89 (s, 2H).

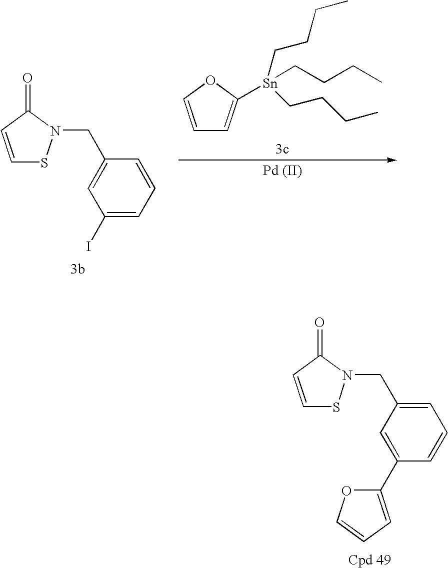

Intermediate Compound 3b (0.51 grams, 1.61 mmol) was dissolved in dioxane (25 mL) and 2-(tributylstannyl)furan Compound 3c (0.65 mL, 2.05 mmol) and dichlorobis(triphenylphosphine) palladium (0.10 grams) was added. The reaction mixture was stirred at 100° C. for 30 min, then cooled to ambient temperature and filtered. The filtrate was concentrated in vacuo and purified on a silica gel flash column. Elution with EtOAc:hexane 2:1 (v/v) gave Compound 49 as a solid. M.P. 78-80° C.; MS 258 (M+H⁺); ¹H NMR (CDCl₃) δ 8.04 (d, 1H), 7.38 (t, 1H), 6.69 (d, 1H), 6.32 (d, 1H), 4.97 (s, 2H).

### EXAMPLE 4

### 2-(3-pyridin-4-yl-benzyl)-isothiazol-3-one (Cpd 50)

Using the procedure of Example 3 and 4-tributylstannanyl-pyridine in place of Compound 3c, Compound 50 was obtained as a solid. MS 269 (M+H⁺); ¹H NMR (CDCl₃) δ 8.76 (d, 1H), 8.67 (d, 1H), 8.09 (d, 1H), 6.33 (d, 1H), 5.04 (s, 2H).

### EXAMPLE 5

### 5-chloro-2-(4-furan-2-yl-benzyl)-isothiazol-3-one (Cpd 23)

Using the procedure of Example 3 and 5-chloro-2-(4-iodobenzyl)isothiazol-3-one Compound 1e in place of Compound 3b, Compound 23 was obtained as a solid. M.P. 100.5-101.8° C.; MS 314 (M+Na); ¹H NMR (CDCl₃) δ 7.77 (d, 2H), 7.47 (s, 1H), 7.32 (d, 2H), 6.67 (d, 1H), 6.48 (t, 1H), 6.30 (s, 1H), 4.88 (s, 2H).

### EXAMPLE 6

### 5-chloro-2-(4-vinyl-benzyl)-isothiazol-3-one (Cpd 27)

Using the procedure of Example 3 and 5-chloro-2-(4-iodobenzyl)isothiazol-3-one Compound 1e in place of Compound 3b and tributyl-vinyl-stannane in place of Compound 3c, Compound 27 was obtained as a solid. ¹H NMR (CDCl3) δ 7.41 (d, 2H), 7.29 (d, 2H), 6.70 (dd, 1H), 6.30 (s, 1H), 5.77 (d, 1H), 5.29 (d, 1H), 4.88 (s, 2H).

### EXAMPLE 7

### 5-chloro-2-[4-(2-methyl-thiazol-4-yl)-benzyl]-isothiazol-3-one (Cpd 31)

Using the procedure of Example 2 and 5-chloro-2-(4-iodobenzyl)isothiazol-3-one Compound 1e in place of Compound 2a, Compound 31 was obtained. ¹H NMR (CDCl₃) δ 7.86 (d, 2H), 7.37 (d, 2H), 7.35 (s, 1H), 6.32 (s, 1H), 4.91 (s, 2H), 2.77 (s, 3H).

### EXAMPLE 8

### 2-[3-(2-methyl-thiazol-4-yl)benzyl]-isothiazol-3-one (Cpd 48)

Using the procedure of Example 2 and 2-(3-iodobenzyl)-isothiazol-3-one Compound 3b in place of Compound 2a, Compound 48 was obtained as a solid. MS 289 (M+H⁺); ¹H NMR (CDCl₃) δ 8.06 (d, 1H), 7.35 (s, 1H), 6.30 (d, 1H), 4.99 (s, 2H), 2.78 (s, 3H).

### EXAMPLE 9

### 4,5-dichloro-2-(4-[1,2,3]thiadiazol-4-yl-benzyl)-isothiazol-3-one (Cpd 24)

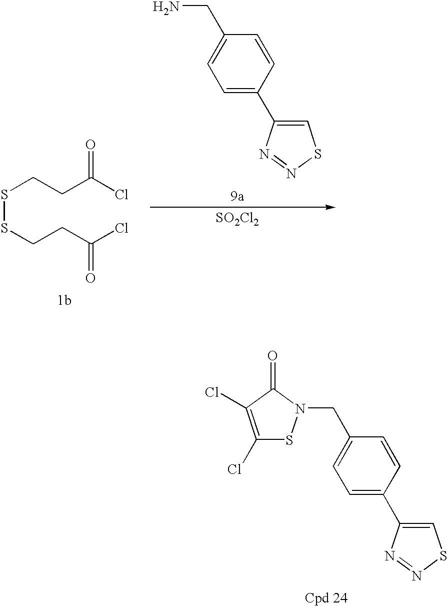

Using the procedure of Example 1, 3-(2-chlorocarbonyl-ethyldisulfanyl)-propionyl chloride Compound 1b (2.31 grams, 10.98 mmol) was placed in a 100 mL round bottom flask. Pyridine (2 drops) was added followed by thionyl chloride (25 mL). The reaction mixture was stirred under nitrogen for 4 hrs, then the solution was evaporated in vacuo. The yellow oil residue was dissolved in DCM (50 mL). DCM (100 mL), 4-(1,2,3-thiadiazol-4-yl)benzylamine hydrochloride Compound 9a (5.00 grams, 21.96 mmol) and TEA (4.59 mL, 32.93 mmol) were added together with stirring in a second flask (300 mL round bottom) cooled in an ice bath. The bis acid chloride solution from the 100 mL flask was added dropwise and the mixture was allowed to stir for 16 hrs. Sulfuryl chloride (2.65 mL, 32.93 mmol) was added and the reaction mixture was stirred for 4 hrs, then the solution was evaporated in vacuo. The crude oil residue was dissolved in DCM (200 mL) and washed with water (200 mL). The organic layer was dried (magnesium sulfate) and concentrated in vacuo. The crude oil was applied to a silica gel flash column and elution with hexane:EtOAc 4:1 (v/v) gave Compound 24 as a tan solid. MS 366 (M+Na); ¹H NMR (CDCl₃) δ 8.70 (s, 1H), 5.03 (s, 2H).

### EXAMPLE 10

### 5-chloro-2-(4-[1,2,3]thiadiazol-4-yl-benzyl)isothiazol-3-one (Cpd 21)

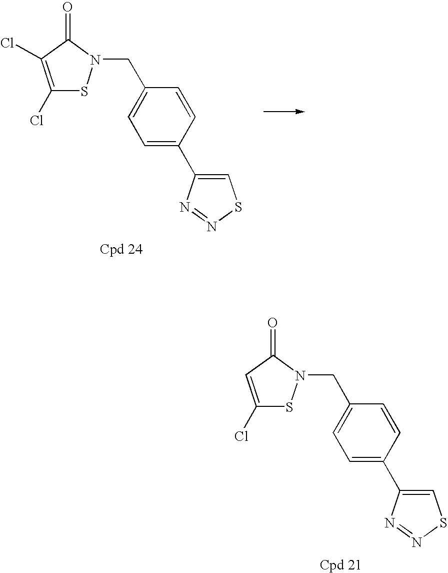

Using the procedure of Example 9, after Compound 24 was eluted, further elution of the same column with hexane:EtOAc 1:1 gave Compound 21 as a tan solid. M.P. 152-154° C.; MS 332 (M+Na); ¹H NMR (CDCl₃) δ 8.68 (s, 1H), 6.33 (s, 1H), 4.97 (s, 2H).

### EXAMPLE 11

### 2-(4-[1,2,3]thiadiazol-4-yl-benzyl)-isothiazol-3-one (Cpd 22)

Using the procedure of Example 9, elution of the crude oil with EtOAc gave Compound 22 as a white solid. MS 298 (M+Na); ¹H NMR (CDCl₃) δ 8.67 (s, 1H), 8.12-8.00 (m, 3H), 6.33 (d, 1H), 5.02 (s, 2H).

Using the foregoing procedures and different starting materials, reagents and conditions as required by those skilled in the art, other compounds representative of the invention may be prepared including, but not limited to,

| Cpd | Compound Name and Properties |
|---|---|
| 29 | 4,5-dichloro-2-(4-methanesulfonyl-benzyl)-isothiazol-3-one |
| | Solid, MS 334(M+H⁺), ¹H NMR(CDCl₃) δ 7.95(d, 2H), 7.50(d, 2H), |
| | 5.00(s, 2H), 3.05(s, 3H). |
| 30 | 4,5-dichloro-2-[1-(4-methanesulfonyl-phenyl)-ethyl]-isothiazol-3-one |
| | Solid, MS 354(M+H⁺), ¹H NMR(CDCl₃) δ 7.98(d, 2H), 7.55(d, 2H), |
| | 5.94(q, 1H), 3.05(s, 3H), 1.90(d, 3H). |
| 33 | 4,5-dichloro-2-(phenyl-pyridin-2-yl-methyl)-isothiazol-3-one |
| | Solid, ¹H NMR(CDCl₃) δ 8.70(d, 1H), 7.75(t, 1H), 7.02(s, 1H), MS |
| | 338.9(M+H⁺). |
| 37 | 4-(5-chloro-3-oxo-3H-isothiazol-2-ylmethyl)-benzoic acid methyl ester |
| | Solid, M.P. 95-96.5° C., ¹H NMR(CDCl₃) δ 8.06(d, 2H), 7.39(d, 2H), |
| | 6.34(s, 1H), 4.95(s, 2H), 3.92(s, 3H). |
| 39 | (1S,2R)-4,5-dichloro-2-(2-phenyl-cyclopropyl)-isothiazol-3-one |
| | Solid, ¹H NMR(CDCl₃) δ 7.35-7.24(m, 5H), 3.15(m, 1H), 2.52(m, 1H), |
| | 1.58(m, 2H). |
| 42 | (1S,2R)-5-chloro-2-(2-phenyl-cyclopropyl)-isothiazol-3-one |
| | Solid, MS 252(M+H⁺), ¹H NMR(CDCl₃) δ 7.34-7.21(m, 5H), 6.23(s, |
| | 1H), 3.05(m, 1H), 2.47(m, 1H), 1.58(m, 2H). |
| 44 | 5-chloro-2-[3-(2-methyl-5-chloro-thiazol-4-yl)-phenyl]-isothiazol-3-one |
| | White solid, M.P. 152-154° C., MS 343(M+H⁺), ¹H NMR(CDCl₃) δ |
| | 6.38(s, 1H), 2.68(s, 3H). |
| 45 | 2-[3-(2-methyl-5-chloro-thiazol-4-yl)-phenyl]-isothiazol-3-one |
| | Tan solid, M.P. 114-117° C., MS 331(M+Na), ¹H NMR(CDCl₃) δ 8.18(d, |
| | 1H), 6.34(d, 1H), 2.68(s, 3H). |
| 46 | 2-[3-(2-methyl-thiazol-4-yl)-phenyl]-isothiazol-3-one |
| | Tan solid, M.P. 108-111° C., MS 275(M+H⁺), ¹H NMR(CDCl₃) δ 8.16(d, |
| | 1H), 7.38(s, 1H), 6.34(d, 1H), 2.78(s, 3H). |
| 47 | 5-chloro-2-[3-(2-methyl-thiazol-4-yl)-phenyl]-isothiazol-3-one |
| | Tan solid, M.P. 125-128° C., MS 309(M+H⁺), ¹H NMR(CDCl₃) δ 7.37(s, |
| | 1H), 6.37(s, 1H), 2.77(s, 3H). |
| 52 | 5-chloro-2-(5-chloro-2-phenyl-thiazol-4-ylmethyl)-isothiazol-3-one |
| | Tan solid, M.P. 143-146° C., MS 343(M+H⁺), 365(M+Na), ¹H |
| | NMR(CDCl₃) δ 6.28(s, 1H), 5.05(s, 2H). |
| 53 | 5-chloro-2-(2-phenyl-thiazol-4-ylmethyl)-isothiazol-3-one |
| | Tan solid, M.P. 130-132° C., MS 309(M+H⁺), ¹H NMR(CDCl₃) δ 7.26(s, |
| | 1H), 6.29(s, 1H), 5.06(s, 2H). |
| 54 | 2-(2-phenyl-thiazol-4-ylmethyl)-isothiazol-3-one |
| | Brown gum, MS 275(M+H⁺), ¹H NMR(CDCl₃) δ 8.08(d, 1H), 7.23(s, |
| | 1H), 6.29(d, 1H), 5.12(s, 2H). |
| 55 | 5-chloro-2-[4-(4-chloro-pyrazol-1-yl)-benzyl]-isothiazol-3-one |
| | Off-white solid, M.P. 163.5-165° C., MS 348(M+Na); ¹H NMR(CDCl₃) δ |
| | 7.9088(s, 1H,), 7.60-7.68(m, 3H,), 6.32(s, 1H), 4.92(s, 2H) |
| 56 | 5-chloro-2-(4-pyrazol-1-yl)-benzyl-isothiazol-3-one |
| | Tan solid, M.P. 130-132° C., MS 292(M+H⁺), 314(M+Na); ¹H |
| | NMR(CDCl₃) δ 7.93(d, 1H), 7.68-7.75(m, 3H), 6.45-6.50(m, 1H), 6.3149(s, |
| | 1H), 4.9185(s, 2H). |
| 57 | 2-[4-(4-chloro-pyrazol-1-yl)-benzyl]-isothiazol-3-one |
| | Tan solid, M.P. 145-147° C. (softens 143), MS 314(M+Na), ¹H |
| | NMR(CDCl₃) δ 8.06(d, 1H), 7.90(s, 1H), 7.59-7.67(m, 3H), 6.32(d, 1H), |
| | 4.97(s, 2H). |
| 58 | 2-(4-pyrazol-1-yl)-benzyl-isothiazol-3-one |
| | Tan solid, M.P. 119-121° C., MS 258(M+H⁺), 280(M+Na), ¹H |
| | NMR(CDCl₃) δ 8.06(d, 1H), 7.92(d, 1H), 7.65-7.74(m, 3H), 6.45-6.50(m, 1H), |
| | 6.31(d, 1H), 4.97(s, 2H). |
| 61 | 5-chloro-2-(5-chloro-2-thiophen-2-yl-thiazol-4-ylmethyl)-isothiazol-3-one |
| | Solid, M.P. 145-147° C., MS 349(M+H⁺), ¹H NMR(CDCl₃) d |
| | 7.48-7.42(m, 2H), 7.11-7.06(m, 1H), 6.28(s, 1H), 5.02(s, 2H). |
| 62 | 5-chloro-(2-thiophen-2-yl-thiazol-4-ylmethyl)-isothiazol-3-one |
| | Brown gum, MS 315(M+H⁺), 1H NMR(CDCl3) d 7.55-7.51(m, 1H), |
| | 7.43-7.40(m, 1H), 7.18(s, 1H), 7.10-7.06(m, 1H), 6.28(s, 1H), 5.02(s, |
| | 2H). |
| 63 | 2-(5-chloro-2-thiophen-2-yl-thiazol-4-ylmethyl)-isothiazol-3-one |
| | Solid, M.P. 137-140° C., MS 315(M+H⁺), ¹H NMR(CDCl₃) d 8.06(d, 1H), |
| | 7.47-7.39(m, 2H), 7.09-7.03(m, 1H), 6.28(d, 1H), 5.06(s, 2H). |
| 64 | 2-(2-thiophen-2-yl-thiazol-4-ylmethyl)-isothiazol-3-one |
| | Gum, MS 281(M+H⁺), 1H NMR(CDCl3) d 8.09(d, 1H), 7.54-7.48(m, |
| | 1H), 7.43-7.38(m, 1H), 7.14(s, 1H)7.12-7.05(m, 1H), 6.29(d, 1H), |
| | 5.08(s, 2H). |

### EXAMPLE 12

### 2-(4-[1,2,3]thiadiazol-4-yl-benzyl)-isothiazol-3-one (Cpd 22)

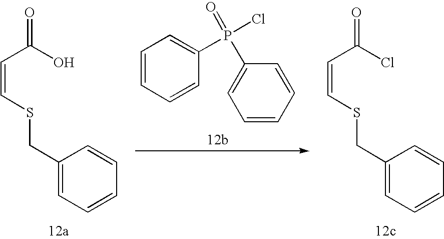

As an alternative method for preparing Compound 22, a published procedure (Beeley et al., A General Synthesis of N-Substituted Isothiazol-3(2H)-ones J. Chem. Soc. Perkin Trans. 1994, 1, 2245-2251) was adapted in which THF (200 mL), acetonitrile (50 mL), (2Z)-3-benzylsulfanyl-acrylic acid Compound 12a (5.00 grams, 25.74 mol) and N-methylmorpholine (9.06 mL, 82.37 mmol) were added to a 500 mL round bottom flask. The mixture was cooled in a sodium chloride-ice water bath, then diphenyl-phosphinic chloride Compound 12b (5.40 mL, 28.31 mmol) was added to provide in situ the (2Z)-3-benzylsulfanyl-acryloyl chloride Compound 12c.

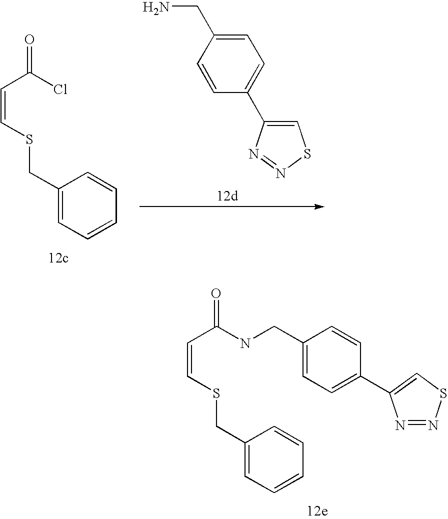

The Compound 12c reaction mixture was stirred for 30 min and 4-[1,2,3]thiadiazol-4-yl-benzylamine hydrochloride Compound 12d (5.86 grams, 25.74 mmol) was added. The reaction mixture was stirred and allowed to warm to ambient temperature for an additional 16 h before the solution was evaporated in vacuo. The crude residue was dissolved in DCM (200 mL) and washed once with water (100 mL). The organic layer was dried (magnesium sulfate) and evaporated to provide 3-benzylsulfanyl-N-(4-[1,2,3]thiadiazol-4-yl-benzyl)-acrylamide Compound 12e as a solid, which was used in the next step without further purification. MS 368 (M+H⁺).

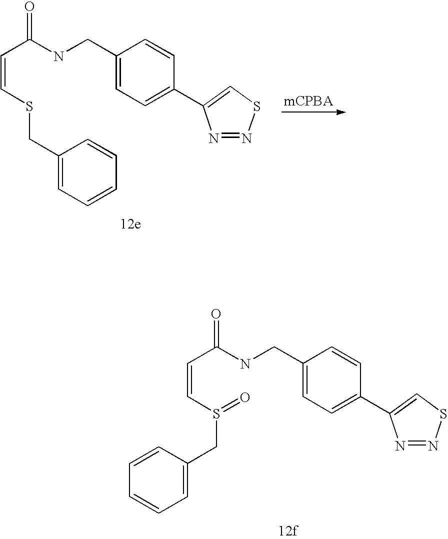

mCPBA (3-chloroperoxybenzoic acid) (5.77 grams, 77%) in DCM (50 mL) was added dropwise to a slurry of Compound 12e in DCM (200 mL) at 0-5° C. The reaction mixture was stirred at 0-5° C. for 30 min, then saturated aqueous sodium hydrogen carbonate (100 mL) was added. The organic layer was dried (sodium sulfate) and evaporated in vacuo to give 3-phenylmethanesulfinyl-N-(4-[1,2,3]thiadiazol-4-yl-benzyl)-acrylamide Compound 12f as a white solid which was used in the next step without further purification. MS 384 (M+H⁺).

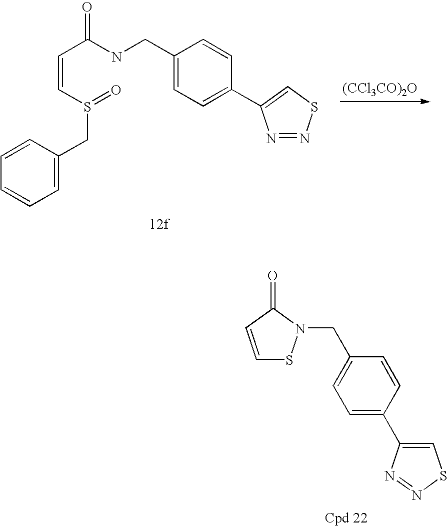

Trichloroacetic anhydride (5.6 mL, 30.9 mmol) was added to a solution of Compound 12f in DCM (250 mL) cooled to 0° C. The reaction mixture was stirred at 0° C. for 1 hr and then at ambient temperature for 1 hr, then washed with water (100 mL). The organic layer was dried ( magnesium sulfate) and evaporated in vacuo to give the crude product which was purified on a silica gel column eluted with EtOAc to provide Compound 22.

### EXAMPLE 13

### 5-chloro-2-(4-benzoylbenzyl)-isothiazol-3-one (Cpd 35)

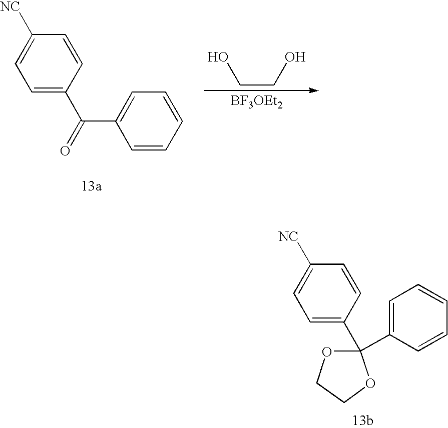

Ethylene glycol (3.30 mL, 59.79 mmol) and boron trifluoride etherate (0.59 mL, 6.38 mmol) were added to a solution of 4-benzoyl-benzonitrile Compound 13a (8.1 g, 39.86 mmol) in benzene (70 mL) at ambient temperature under N₂. The flask was equipped with a Dean-Stark trap and the resulting solution was heated to reflux for 24 hrs. After cooling to ambient temperature, the reaction mixture was washed with saturated aqueous sodium bicarbonate and then with brine. The organic layer was dried (magnesium sulfate), then filtered, concentrated and dried under vacuum to provide 4-(2-phenyl-[1,3]dioxolan-2-yl)-benzonitrile Compound 13b which was not isolated. The residue was dissolved in THF (50 mL) and cooled to 0° C. (ice bath).

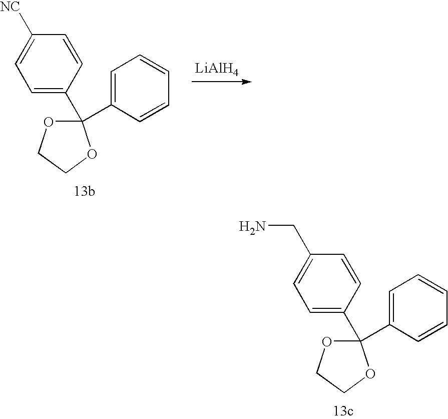

LAH (1 N in THF, 80 mL) was added over a 15 min period to the solution of Compound 13b. The ice bath was removed and the reaction mixture was stirred and allowed to warm to room temperature for 1.5 h. The solution was cooled again using an ice bath and carefully quenched with 5% sodium hydroxide solution. The THF was evaporated in vacuo, then chloroform (100 mL) was added to the residue and the resulting mixture was filtered. The filtrate was washed with saturated aqueous sodium bicarbonate solution, then brine, and dried (magnesium sulfate). The residue was filtered again, then concentrated and dried in vacuo to provide 4-(2-phenyl-[1,3]dioxolan-2-yl)-benzylamine Compound 13c (7.5 g, 74%) as a clear liquid which was used in the next step without further purification. ¹H NMR (CDCl₃) δ 7.48-7.53(m, 4H), 7.26-7.35 (m, 5H), 4.05 (s, 4H), 3.84 (s, 2H). MS 256.0 (M+H⁺).

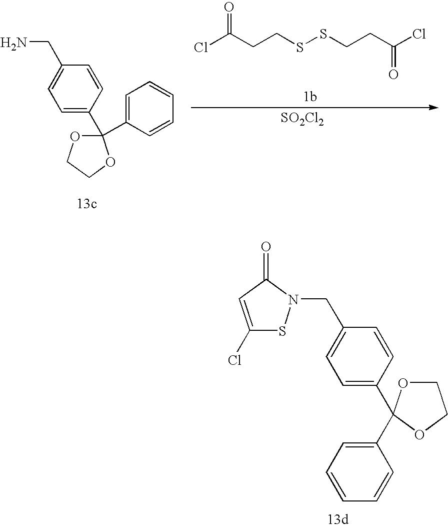

Using the procedure of Example 1 and Compound 13c in place of Compound 1C, intermediate 2-[4-(2-phenyl-[1,3]dioxolan-2-yl)-benzyl]-isothiazol-3-one Compound 13d was obtained.

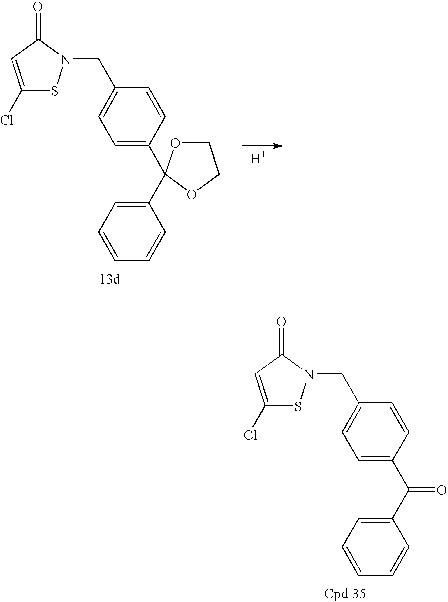

Hydrochloric acid (1N in diethyl ether, 6 mL) was added to a solution of Compound 13d (0.43 grams, 1.15 mmol) in DCM (10 mL). The reaction mixture was stirred under nitrogen at ambient temperature for 12 hrs, then the solution was evaporated in vacuo. The product was purified using a silica gel column eluted with EtOAc:hexane 1:1 (v/v) to provide Compound 35 (0.27 grams, 71%) as a white solid. ¹H NMR (CDCl₃) δ 7.85-7.78 (m, 4H), 7.60 (t, 1H), 7.49 (t, 2H), 7.42 (d, 2H), 6.44 (s, 1H), 4.97 (s, 2H), MS 352 (M+Na).

Using the procedure of Example 13 and different starting materials, reagents and conditions as required by those skilled in the art, other compounds representative of the invention may be prepared including, but not limited to,

| Cpd | Compound Name and Properties |
|---|---|
| 34 | 4,5-dichloro-2-(4-benzoylbenzyl)-isothiazol-3-one |
| | ¹H NMR(CDCl₃) δ 7.82-7.78(m, 4H), |
| | 7.60(t, 1H), 7.50(t, 2H), 7.42(d, 2H), |
| | 5.02(s, 2H), MS 364(M+H⁺) |
| 51 | 2-(4-benzoylbenzyl)-isothiazol-3-one |
| | ¹H NMR(CDCl₃) δ 8.53(d, 1H), |
| | 7.73-7.64(m, 5H), 7.55(t, 2H), 7.41(d, |
| | 2H), 6.27(d, 1H), 5.04(s, 2H) |

### EXAMPLE 14

### 5-chloro-2-(3-[1,2,3]thiadiazol-4-ylbenzyl)-isothiazol-3-one (Cpd 32)

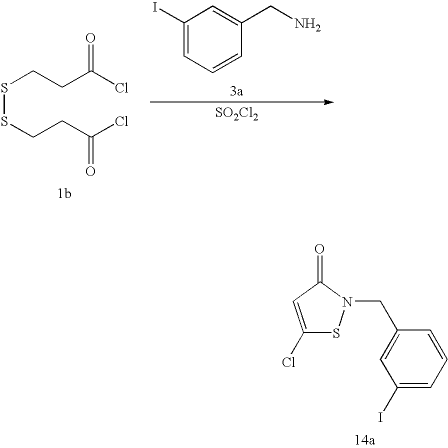

Using the procedure of Example 1 and 3-iodobenzylamine Compound 3a in place of Compound 1c, intermediate 5-chloro-2-(3-iodobenzyl)-isothiazol-3-one Compound 14a was obtained as a white solid. M.P. 84-86° C., ¹H NMR (CDCl₃) δ 7.11 (t, 1H), 6.30 (s, 1H), 4.84 (s, 2H).

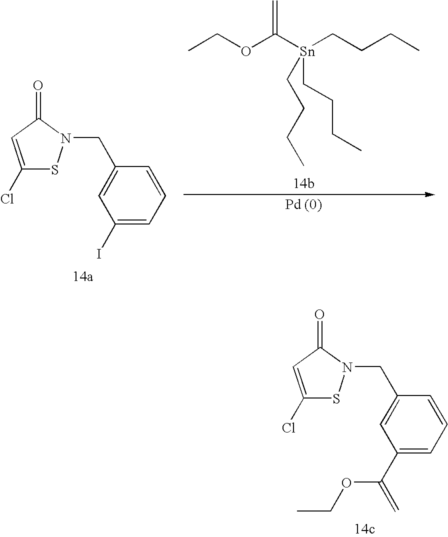

Tributyl(1-ethoxyvinyl)tin Compound 14b (2.46 grams, 6.82 mmol) and tetrakis(triphenylphosphine) palladium (0) 0.39 grams, 0.34 mmol) were added to a solution of Compound 14a (1.20 grams, 3.41 mmol) in dioxane (30 mL). The reaction mixture was stirred at reflux for 3 hrs and the solution was evaporated in vacuo to provide 5-chloro-2-[3-(1-ethoxy-vinyl)-benzyl]-isothiazol-3-one Compound 14c as a residue which was not isolated.

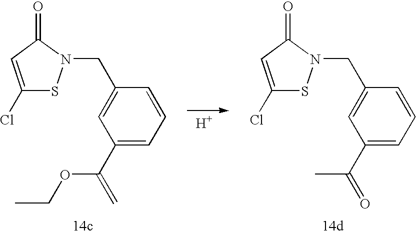

The Compound 14c residue was dissolved in methanol (30 mL), water (8 mL) and 1N HCl (3.5 mL). The mixture was stirred for 1 hr, then neutralized with saturated aqueous sodium bicarbonate. The product was extracted with DCM and purified using column chromatography (silica gel) eluted with EtOAc:hexane 1:1 (v/v) to provide 2-(3-acetyl-benzyl)-5-chloro-isothiazol-3-one Compound 14d (0.44 grams, 48%) as an oil. ¹H NMR (CDCl₃) δ 7.95-7.90 (m, 2H), 7.65-7.46 (m, 2H), 6.31 (s, 1H), 4.94 (s, 2H), 2.62 (s, 3H).

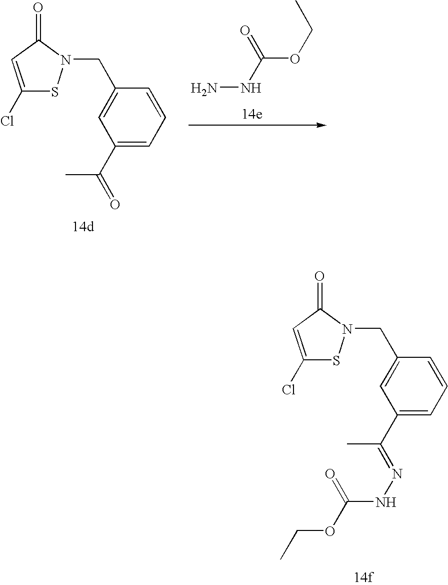

Hydrazinecarboxylic acid ethyl ester Compound 14e (0.16 grams, 1.56 mmol) and p-toluenesulfonic acid (10 mg) were added to a solution of Compound 14d (0.40 grams, 1.49 mmol) in toluene (20 mL). The reaction mixture was stirred at reflux for 3 hrs with azeotropic removal of water. The toluene was evaporated in vacuo to provide N′-{1-[3-(5-chloro-3-oxo-3H-isothiazol-2-ylmethyl)-phenyl]-ethylidene}-hydrazinecarboxylic acid ethyl ester Compound 14f as a residue which was not isolated.

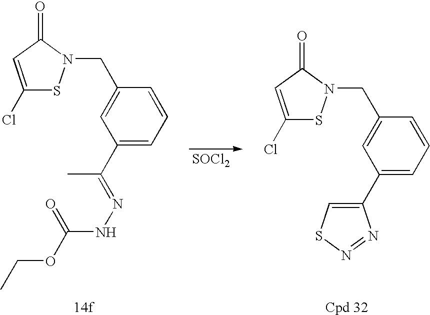

The Compound 14f residue was dissolved in thionyl chloride (20 mL) and the reaction mixture was stirred at 60° C. for 1 hr. The thionyl chloride was evaporated in vacuo and the crude product was chromatographed on silica gel eluted with EtOAc:hexane 1:1 (v/v) to provide Compound 32 (0.16 grams, 35%) as a pale yellow powder. 1H NMR (8.67 (s, 1H), 8.02-7.97 (m, 2H), 7.52 (t, 1H), 7.33 (d, 1H), 6.32 (s, 1H), 4.98 (s, 2H).

### EXAMPLE 15

### 5-chloro-2-(4-ethynyl-benzyl)-isothiazol-3-one (Cpd 40)

### 3-[4-(5-chloro-3-oxo-3H-isothiazol-2ylmethyl)-phenylethynyl]-benzonitrile (Cpd 43)

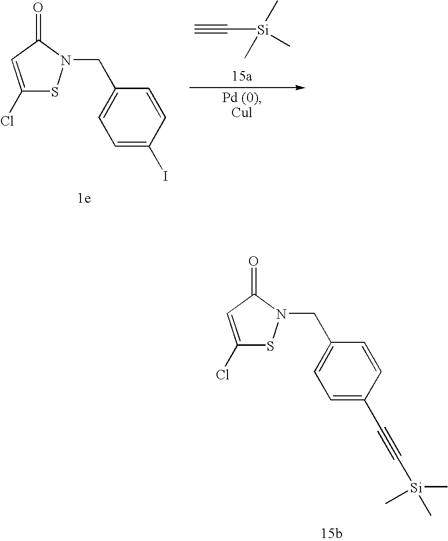

Ethynyltrimethylsilane Compound 15a (2.80 mL, 19.89 mmol) was added in 3 portions over a 1 hr period to a mixture of 5-chloro-2-(4-iodobenzyl)-isothiazol-3-one Compound 1e (2.33 grams, 6.63 mmol), tetrakis(triphenylphosphine) palladium (0) (0.15 grams, 2 mol %) copper (I) iodide (0.05 grams, 4 mol %) and diisopropylamine (25 mL) in toluene (130 mL). The reaction mixture was stirred for 12 hrs, then washed with water. The aqueous layer was extracted with DCM and the organic layers were combined and dried (magnesium sulfate), then concentrated in vacuo and purified on a silica gel column using a mobile phase of EtOAc:hexane 1:2 to give 5-chloro-2-(4-trimethylsilanylethynyl-benzyl)-isothiazol-3-one Compound 15b (1.63 grams, 76%) as a white powder. ¹H NMR (CDCl₃) δ 7.46 (d, 2H), 7.22 (d, 2H), 6.30 (s, 1H), 4.88 (s, 2H), 0.25 (s, 9H).

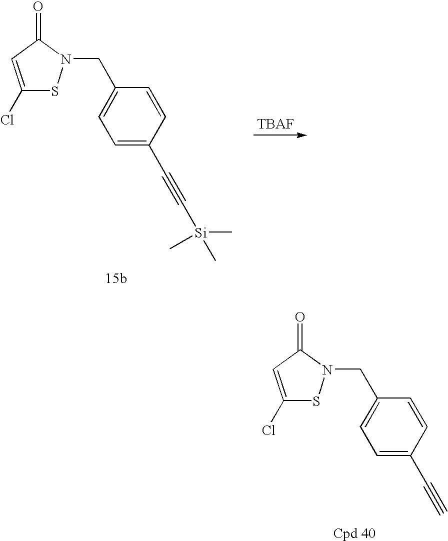

TBAF (tetrabutylammonium fluoride) (5.3 mL, 1N solution in THF) was added dropwise to an ice-bath cooled solution of Compound 15b (1.63 grams, 5.09 mmol) in DCM (30 mL). The reaction mixture was stirred for 5 min the water (30 mL) was added. The aqueous layer was separated and extracted once with DCM (50 mL). The combined organic layers were dried (magnesium sulfate), concentrated and applied to a silica gel column eluted with EtOAc:hexane 1:2 to provide Compound 40 (0.40 grams, 31%) as a white powder. ¹H NMR (CDCl₃) δ 7.58 (d, 2H), 7.23 (d, 2H), 6.30 (s, 1H), 4.90 (s, 2H), 3.09 (s, 1H), MS 250 (M+H⁺).

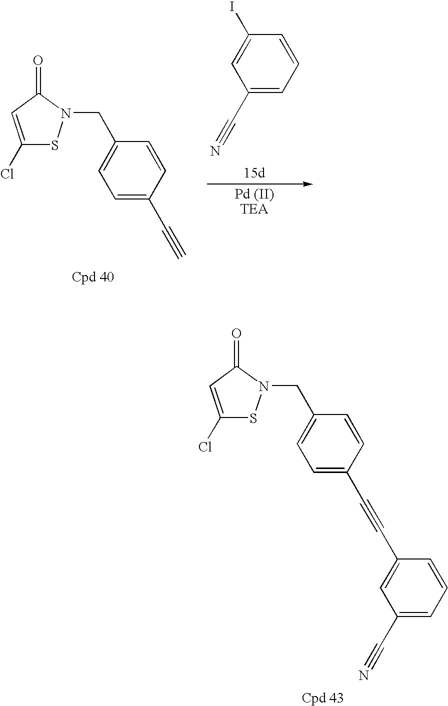

A mixture of Compound 40 (0.031 grams, 0.124 mmol), 3-iodobenzonitrile Compound 15d (0.17 grams, 0.744 mmol) and bis(acetato) bis(triphenylphosphine) palladium (II) (0.010 gram) in triethylamine (0.50 mL) was heated to 70° C. for 3 hrs. The reaction mixture was directly applied to a silica gel column and eluted with EtOAc:hexane 1:1 to afford Compound 43 (0.021 grams, 48%) as a white powder. 1H NMR (CDCl3) d 7.78 (s, 1H), 7.72 (d, 1H), 7.58 (d, 1H), 7.49 (m, 2H), 7.45 (t, 1H), 7.27 (m, 2H), 6.28 (s, 1H), 4.90 (s, 2H).

Using the procedure of Example 15 and different starting materials, reagents and conditions as required by those skilled in the art, other compounds representative of the invention may be prepared including, but not limited to,

| Cpd | Compound Name and Properties |
|---|---|
| 28 | 4,5-dichloro-2-[4-(3-chlorophenylethynyl)-benzyl]-isothiazol-3-one |
| | ¹H NMR(CDCl₃) δ 7.55(m, 3H), 7.41(m, 1H), |
| | 7.36-7.26(m, 4H), 4.96(s, 2H), MS 391(M+H⁺). |

### EXAMPLE 16

### 5-chloro-2-(4-benzenesulfonyl-benzyl)-isothiazol-3-one (Cpd 38)

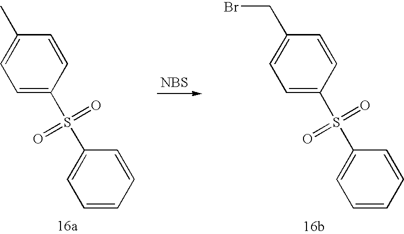

Dibenzoyl peroxide (71.8 mg) and NBS (N-bromosuccinimide) (3.29 grams, 18.49 mmol) were added to a solution of 4-phenyl-p-tolylsulfone Compound 16a (3.58 grams, 15.41 mmol) in carbon tetrachloride (75 mL). The reaction mixture was heated to reflux and stirred for 30 min., then filtered. The filtrate was concentrated in vacuo and chromatographed on silica gel eluted with a hexane:EtOAc 9:1 mobile phase to provide 1-bromomethyl-4-phenylsulfonyl-benzene Compound 16b (82%).

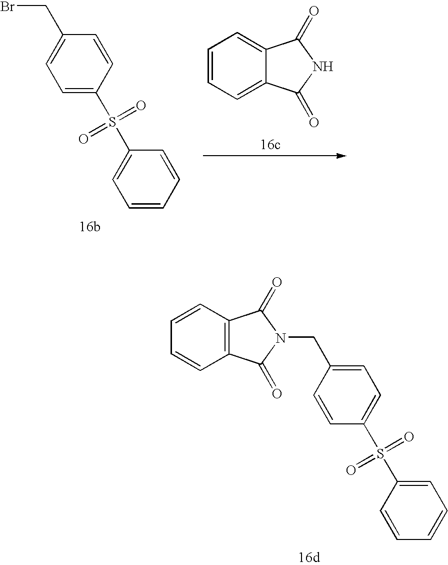

A mixture of Compound 16b (5.00 grams, 16.07 mmol), phthalimide Compound 16c (2.60 grams, 17.67 mmol) and potassium carbonate (2.66 grams, 19.28 mmol) in anhydrous DMF (100 mL) was stirred at 70° C. for 3 hrs, then water (200 mL) was added and the product was extracted with EtOAc. The organic layer was washed once with water, dried (magnesium sulfate) and evaporated to give a crude solid which was triturated with DCM and filtered. The filtrate was concentrated and chromatographed on a silica gel column eluted with hexane:EtOAc 3:2 to provide 2-(4-benzenesulfonyl-benzyl)-isoindole-1,3-dione Compound 16d (90%) as a white solid.

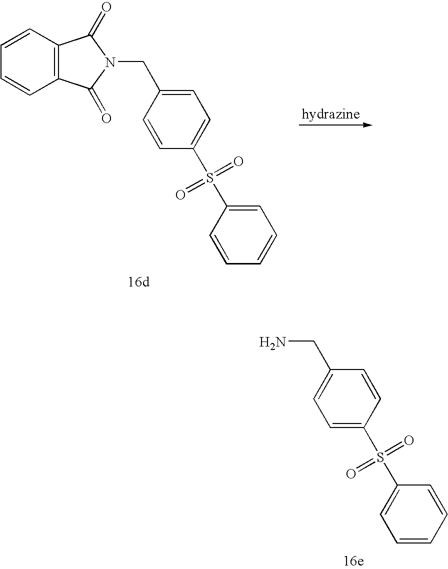

A suspension of Compound 16d (1.56 grams, 4.14 mmol) in absolute ethanol (25 mL) was treated with hydrazine hydrate (0.52 mL, 16.56 mmol). The reaction mixture was stirred at reflux for 3 hrs. The solution was evaporated in vacuo and the crude product was dissolved in DCM (100 mL) and washed with 10% aqueous sodium hydroxide (100 mL). The aqueous layer was washed with DCM (50 mL) and the organic layers were combined and washed with water, dried (magnesium sulfate) and filtered. Evaporation of the filtrate gave 4-benzenesulfonyl-benzylamine Compound 16e (94%) as a-white solid. ¹H NMR (DMSO d₆) δ 7.92-7.86 (m, 4H), 7.60-7.51 (m, 5H), 3.92 (s, 2H), MS 248 (M+H⁺).

Using the procedure of Example 1 and Compound 16e in place of Compound 1c, Compound 38 was obtained. ¹H NMR (CDCl₃) δ 7.95 (d, 4H), 7.59-7.49 (m, 3H), 7.40 (m, 2H), 6.31 (s, 1H), 4.93 (s, 2H), MS 387.9 (M+H⁺).

### EXAMPLE 17

### 5-chloro-2-[4-(pentane-1-sulfonyl)-benzyl]-isothiazol-3-one (Cpd 41)

Using the procedure of Example 16 to prepare 4-(pentane-1-sulfonyl)-benzylamine Compound 17a, then using the procedure of Example 1 and Compound 17a in place of Compound 1c, Compound 41 was obtained. ¹H NMR (CDCl₃) δ 7.91 (d, 2H), 7.48 (d, 2H), 6.34 (s, 1H), 4.98 (s, 2H), 3.09-3.05 (m, 2H), 1.75-1.67 (m, 2H), 1.38-1.26 (m, 4H), 0.86 (t, 3H), MS 360.0 (M+H⁺).

### EXAMPLE 18

### 5-chloro-2-[4-(4-fluoro-benzenesulfonyl)-benzyl]-isothiazol-3-one (Cpd 36)

Using the procedure of Example 16 to prepare 4-(4-fluoro-benzenesulfonyl)-benzylamine Compound 18a, then using the procedure of Example 1 and Compound 18a in place of Compound 1c, Compound 36 was obtained. ¹H NMR (CDCl₃) δ 7.97-7.90 (m, 4H), 7.42 (m, 2H), 7.21-7.16 (m, 2H), 6.31 (s, 1H), 4.92 (s, 2H), MS 405.9 (M+H⁺).

### EXAMPLE 19

### 5-chloro-2-(3-formylbenzyl)-isothiazol-3-one (Cpd 59)

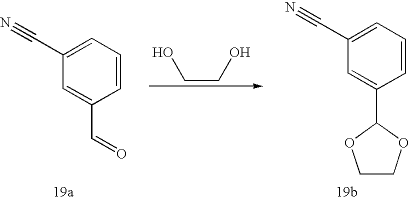

3-cyanobenzaldehyde Compound 19a (10.00 grams, 76.3 mmol), p-toluenesulfonic acid (2.0 grams), ethylene glycol (18 mL) and benzene (100 mL) were added to a 200 mL round bottom flask equipped with a Dean-Stark trap. The reaction mixture was stirred at reflux for 18 hrs before being concentrated in vacuo. EtOAc (200 mL) was added and the mixture was washed with saturated aqueous sodium bicarbonate, then water. The organic layer was dried (sodium sulfate) and evaporated to give 3-[1,3]dioxolan-2-yl-benzonitrile Compound 19b (12.43 g, 93%) as an oil. ¹H NMR (CDCl₃) δ 7.80 (s, 1H), 7.70 (d, 1H), 7.65 (d, 1H), 7.50 (t, 1H), 5.83 (s, 1H), 4.10 (m, 4H).

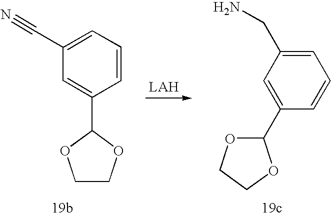

Compound 19b (7.82 grams, 45.0 mmol) was dissolved in THF (50 mL) and added dropwise with ice bath cooling to 100 mL of 1N solution of LAH in THF. The reaction mixture was stirred for 2 hrs then quenched with water. The THF was evaporated in vacuo and the resulting residue was dissolved in chloroform (200 mL) and washed with water, then dried (sodium sulfate) and evaporated to give 3-[1,3]dioxolan-2-yl-benzylamine Compound 19c (5.53 g, 69%) as an oil. ¹H NMR (CDCl₃) δ 7.43 (s, 1H), 7.34 (m, 3H), 5.81 (s, 1H), 4.10 (m, 4H), 3.88 (s, 2H).

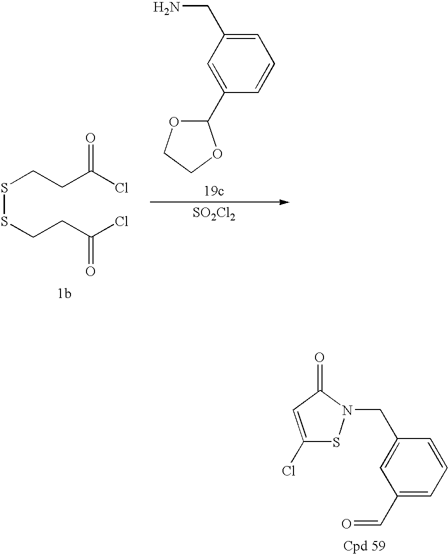

3-(2-chlorocarbonyl-ethyldisulfanyl)-propionyl chloride Compound 1b (3.15 grams, 14.98 mmol) was stirred in thionyl chloride (20 mL) at ambient temperature for 16 hrs, then the solution was evaporated in vacuo. The resulting oil was dissolved in DCM (40 mL) and again evaporated in vacuo to a yellow oil which was transferred dropwise to a second ice bath cooled flask containing DCM (100 mL), TEA (4.2 mL, 30.1 mol) and Compound 19c (5.53 grams, 31 mmol). The reaction mixture was stirred for 1 hr, then sulfuryl chloride (4.8 mL, 60 mmol) was added. The mixture was stirred for 3 hrs, then the solution was evaporated in vacuo. The residue was again dissolved in DCM (200 mL) and washed with water. The organic layer was dried (sodium sulfate) and concentrated in vacuo. The crude oil was column chromatographed on silica gel and eluted with hexane:EtOAc 1:1 to afford Compound 59 as an oil. ¹H NMR (CDCl₃) δ 10.03 (s, 1H), 6.33 (s, 1H), 4.96 (s, 2H).

### EXAMPLE 20

### [3-(5-chloro-3-oxo-3H-isothiazol-2-ylmethyl)-phenyl]-propynal (Cpd 60)

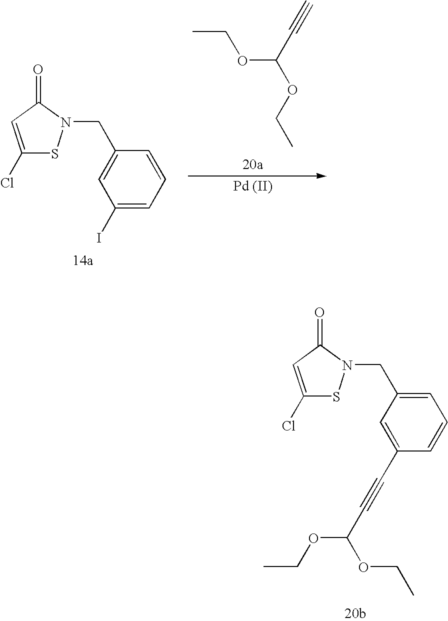

TEA (2.00 mL), propionlaldehyde diethylacetal Compound 20a (1.00 mL, 6.98 mmol) and bis (triphenylphosphine) palladium dichloride (0.21 grams) were added to a solution of 5-chloro-2-(3-iodobenzyl)-isothiazol-3-one Compound 14a (1.16 grams, 3.30 mmol) in acetonitrile (50 mL). The reaction mixture was stirred at reflux for 30 min, the solution was evaporated in vacuo to provide 5-chloro-2-[3-(3,3-diethoxy-prop-1-ynyl)-benzyl]-isothiazol-3-one Compound 20b as a crude red oil which was not isolated.

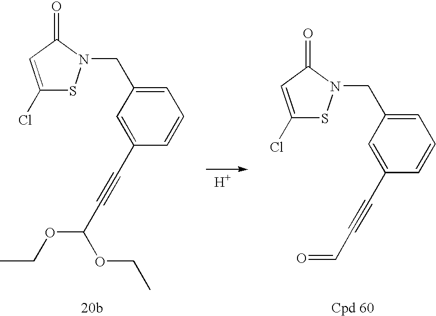

Compound 20b was dissolved in a mixture of THF (25-mL), water (25 mL) and sulfuric acid (1 mL). The reaction mixture was stirred at reflux for 30 min, then EtOAc (50 mL) was added and the layers were separated. The organic layer was washed with saturated aqueous sodium bicarbonate, then with brine. The solution was evaporated in vacuo and the product was purified on a silica gel column eluted with EtOAc:hexane 1:1 to provide Compound 60 as a solid. ¹H NMR (CDCl₃) δ 9.43 (s, 1H), 7.56 (m, 2H), 7.45 (m, 2H), 6.33 (s, 1H), 4.90 (s, 2H).

### EXAMPLE 21

### 4,5-dichloro-2-(4-acetylbenzyl)-isothiazol-3-one (Cpd 65)

Using the procedure of Example 2 and Compound 1d in place of Compound 2a, Compound 65 was obtained as a brown solid. ¹H NMR (CDCl₃) δ 7.95 (d, 2H), 7.43 (d, 2H), 5.01 (s, 2H), 2.60 (s, 3H).

### EXAMPLE 22

### 5-chloro-2-naphthalen-1-ylmethyl-isothiazol-3-one (Cpd 1)

### 2-naphthalen-1-ylmethyl-isothiazol-3-one (Cpd 2)

Using the procedure of Example 1 and 1-naphthylenemethylamine Compound 22a (2.24 mL, 15.3 mmol) in place of Compound 1c, Compound 1 was obtained as an off white solid. M.P. 131.5-133.5° C.; ¹H NMR (CDCl₃) δ 6.29 (s, 1H), 5.35 (s, 2H).

Further elution of the same column with EtOAc gave Compound 2 as a tan solid. M.P. 121-125° C.; ¹H NMR (CDCl₃) δ 8.09 (d, 1H), 6.29 (d, 1H), 5.38 (s, 2H).

Using the procedure of Example 22 and different starting materials, reagents and conditions as required by those skilled in the art, other compounds representative of the invention may be prepared including, but not limited to,

| Cpd | Compound Name and Properties |
|---|---|
| 5 | 5-chloro-2-(1-naphthalen-1-yl-ethyl)-isothiazol-3-one |
| | Oil, ¹H NMR(CDCl₃) δ 8.06(d, 1H), 7.90(t, 2H), |
| | 7.68(d, 1H), 7.55(m, 4H), 6.57(q, 1H), 6.25(s, 1H), 1.87(d, 3H). |
| 13 | 2-naphthalen-2-ylmethyl-isothiazol-3-one |
| | Solid, MS 242(MH⁺); ¹H NMR(CDCl₃) δ 8.04(d, 1H), |
| | 6.32(s, 1H), 5.10(s, 2H). |
| 14 | 5-chloro-2-naphthalen-2-ylmethyl-isothiazol-3-one |
| | Solid, M.P. 104.5-106.5° C.; MS 276(MH⁺); ¹H NMR(CDCl₃) |
| | δ 6.32(s, 1H), 5.05(s, 2H). |
| 15 | 4,5-dichloro-2-naphthalen-2-ylmethyl-isothiazol-3-one |
| | Solid, M.P. 101-103° C.; MS 332(M+Na); |
| | ¹H NMR(CDCl₃) δ 5.12(s, 2H). |

### EXAMPLE 23

### 5-chloro-2-indan-1-yl-isothiazol-3-one (Cpd 3)

Using the procedure of Example 1 and indan-1-ylamine Compound 23a (2.00 mL, 16.0 mmol) in place of Compound 1c, Compound 3 was obtained as an oil. ¹H NMR (CDCl₃) δ 7.25 (m, 4H) 6.28 (s, 1H), 6.08 (t, 1H), 3.10 (m, 1H), 2.95 (m, 1H), 2.65 (m, 1H), 2.09 (m, 1H).

Using the procedure of Example 23 and different starting materials, reagents and conditions as required by those skilled in the art, other compounds representative of the invention may be prepared including, but not limited to,

| Cpd | Compound Name and Properties |
|---|---|
| 4 | 5-chloro-2-(1,2,3,4-tetrahydro-naphthalen-1-ylmethyl)-isothiazol- |
| | 3-one Solid, M.P. 84-85° C.; ¹H NMR(CDCl₃) |
| | δ 7.15(m, 4H), 6.28(s, 1H), 5.77(t, 1H). |

### EXAMPLE 24

### 5-chloro-2-(3-chloro-benzo[b]thiophen-2-ylmethyl)-isothiazol-3-one (Cpd 6)

### 2-benzo[b]thiophen-2-ylmethyl-5-chloro-isothiazol-3-one (Cpd 7)

### 2-(3-chloro-benzo[b]thiophen-2-ylmethyl)-isothiazol-3-one (Cpd 8)

### 2-benzo[b]thiophen-2-ylmethyl-isothiazol-3-one (Cpd 9)

Using the procedure of Example 1 and 2-benzo[b]thiophene methanamine Compound 24a (5.00 g, 30.6 mmol) in place of Compound 1c, elution with hexane:EtOAc 4:1 afforded Compound 6 as a tan solid. M.P. 118-120° C.; MS 338 (M+Na); ¹H NMR (CDCl₃) δ 6.29 (s, 1H), 5.21 (s, 2H). Further elution of the same column with hexane:EtOAc 1:1 gave Compound 7 and Compound 8.

Compound 7 was obtained as a tan solid. M.P. 101-103° C.; MS 304 (M+Na); ¹H NMR (CDCl₃) δ 7.31 (s, 1H), 6.30 (s, 1H), 5.14 (s, 2H). Compound 8 was obtained as a tan solid. M.P. 139-142° C.; MS 282 (MH⁺); ¹H NMR (CDCl3) δ 8.06 (d, 1H), 6.30 (d, 1H), 5.26 (s, 2H). Final elution with EtOAc gave Compound 9 as a tan solid. M.P. 144-146° C.; MS 270 (M+Na); ¹H NMR (CDCl₃) δ 8.07 (d, 1H), 6.30 (d, 1H), 5.19 (s, 2H).

Using the procedure of Example 24 and different starting materials, reagents and conditions as required by those skilled in the art, other compounds representative of the invention may be prepared including, but not limited to,

| Cpd | Compound Name and Properties |
|---|---|
| 10 | 2-benzo[b]thiophen-3-ylmethyl-4,5-dichloro-isothiazol-3-one |
| | Yellow gum, MS 338(M+Na); ¹H NMR(CDCl₃) δ 7.56(s, 1H), |
| | 5.23(s, 2H). |
| 11 | 2-benzo[b]thiophen-3-ylmethyl-5-chloro-isothiazol-3-one |
| | Off white solid, M.P. 98-100° C.; MS 304(M+Na); |
| | ¹H NMR(CDCl₃) δ 7.50(s, 1H), 6.29(s, 1H), |
| | 5.16(s, 2H). |
| 12 | 2-benzo[b]thiophen-3-ylmethyl-isothiazol-3-one |
| | Tan solid, M.P. 143-145° C.; MS 270(M+Na); ¹H NMR(CDCl₃) |
| | δ 7.99(d, 1H), 7.48(s, 1H), 6.29(d, 1H), 5.19(s, 2H). |
| 18 | 4,5-dichloro-2-(5-chloro-benzo[b]thiophen-3-ylmethyl)-isothiazol- |
| | 3-one Tan solid, M.P. 151-152° C.; MS 372(M+Na); |
| | ¹H NMR(CDCl₃) δ 7.61(s, 1H), 5.17(s, 2H). |
| 19 | 5-chloro-2-(5-chloro-benzo[b]thiophen-3-ylmethyl)-isothiazol- |
| | 3-one Pale yellow solid, M.P. 125-127° C.; MS 338(M+Na); |
| | ¹H NMR(CDCl₃) δ 7.54(s, 1H), 6.32(s, 1H), 5.11(s, 2H). |
| 20 | 2-(5-chloro-benzo[b]thiophen-3-ylmethyl)-isothiazol-3-one |
| | Tan solid, M.P. 121-123° C.; MS 282(MH⁺); |
| | 1H NMR(CDCl3) d 8.03(d, 1H), 7.53(s, 1H), 6.32(d, 1H), |
| | 5.15(s, 2H). |

### EXAMPLE 25

### 5-chloro-2-(2,3-dihydro-benzofuran-5-ylmethyl)-isothiazol-3-one (Cpd 16)

### 2-(2,3-dihydro-benzofuran-5-ylmethyl)-isothiazol-3-one (Cpd 17)

Using the procedure of Example 1 and 5-(aminomethyl)-2,3-dihydrobenzo[b]furan Compound 25a (3.19 g, 21.4 mmol) in place of Compound 1c, elution with hexane:EtOAc 1:1 afforded Compound 16 as a tan solid. M.P. 100-102° C.; MS 290 (M+Na), ¹H NMR (CDCl₃) δ 6.28 (s, 1H), 4.80 (s, 2H), 4.59 (t, 2H), 3.21 (t, 2H).

Further elution of the same column with EtOAc afforded Compound 17 as a brown gum. MS 256 (M+Na); ¹H NMR (CDCl₃) δ 8.03 (d, 1H), 6.32 (d, 1H), 4.86 (s, 2H), 4.58 (t, 2H), 3.20 (t, 2H).

Additional compounds may be made according to the synthetic methods of the present invention by one skilled in the art, differing only in possible starting materials, reagents and conditions used in the instant methods.

### Biological Procedures

The following examples illustrate that the compounds of the invention are useful in a method for preventing, treating or ameliorating a cPLA₂ mediated inflammatory related disease, disorder or condition and, more particularly, for preventing, treating or ameliorating a cPLA₂ mediated inflammatory related disease, disorder or condition which results from the cellular secretion of TXB₂ or LTB₄.

PMA Induced Acute Ear Edema

The mouse acute ear edema model demonstrates the ability of the compounds of the present invention to inhibit PMA (phorbol myristate acetate) induced inflammation.

PMA was prepared 200 ng/mL in an acetone:water mixture (99:1). 10 μL of 200 ng/10 μL was applied to the left ears of the mice. Control mice received 10 μL of vehicle. Test compounds were dissolved in the vehicle and applied at a test concentration of from about 1% to about 10% (depending on solubility) at 10 min before PMA application. β-methasone (1%) was used as a control. Six hours after the PMA application, mice were sacrificed and 6 mm ear punch biopsies were removed and weighed to assess edema. The percent inhibition of edema for compounds tested is shown in Table 1.

TABLE 1

| Cpd | % Inh | β-methasone |
|---|---|---|
| 9 | 72% | 100% |
| 51 | 84% | 100% |
| 57 | 100% | 100% |

Neutrophil LTB₄ Assay

Neutrophils purified from human blood were resuspended in HBSS buffer containing calcium and magnesium to a cell density of 1×10⁶/mL. Purified neutrophils were then incubated with test compounds at a test concentration of 5 μM for 15 min at 37° C. Following incubation, the cells were challenged with calcium ionophore (1 μM final) for 15 minutes at 37° C. Negative control wells were incubated with equal volume of vehicle. The reaction was stopped by transferring the plate to ice for 5 minutes. The plate was centrifuged at 1500 RPM for 5 min at 4° C. LTB₄ was calculated in cell free supernatants by EIA (Assay Design Inc). The percent inhibition at the test concentration of 5 μM or the IC₅₀ (μM) for compounds tested is shown in Table 2.

Ionophore and Arachidonic Acid Induced TXB₂ Released by Human Platelets

Aliquots of Human Platelet-rich plasma (PRP), diluted to 1:25 with 0.9% NaCl containing 14.4 U heparin/mL, were incubated with test compounds at a test concentration of 1 μM. The plate was incubated for 15 minutes at 37° C. Following incubation, platelets were challenged with calcium ionophore (final 10 μM) or arachidonic acid (AA) (final 50 μM) and the plate was further incubated at 37° C. for 15 min. The plate was transferred to an ice bed for 5 min in order to stop the reaction. Supernatants were assayed for TXB₂ production by EIA (Assay Design Inc) after diluting with assay buffer (Assay Design Inc). The percent inhibition at the test concentration of 1 μM or the IC₅₀ (μM) for compounds tested is shown in Table 2.

TABLE 2

| Cpd | TXB₂ | LTB₄ |
|---|---|---|
| 1 | 3 | 99% |
| 2 | 3 | 81% |
| 3 | 0.9 | 86% |
| 4 | 3.3 | 100% |
| 5 | 0.6 | 100% |
| 6 | 0.71 | 70% |
| 7 | 0.46 | 95% |
| 8 | 1.4 | 74% |
| 9 | 1.8 | 84% |
| 10 | 84% | 100% |
| 11 | 68% | 100% |
| 12 | 0.75 | 89% |
| 13 | 1.3 | 78% |
| 14 | 0.35 | 96% |
| 15 | 0.33 | 75% |
| 16 | 0.25 | 97% |
| 17 | 5.8 | 39% |
| 18 | 0.28 | 71% |
| 19 | 1 | 100% |
| 20 | 1 | 88% |
| 21 | 1 | 87% |
| 22 | 1.2 | 50% |
| 23 | 0.3 | 100% |
| 24 | 0.5 | 52% |
| 25 | 1 | 54% |
| 26 | 0.4 | 1.7 μM |
| 27 | 0.8 | 100% |
| 28 | 3.5 | 100% |
| 29 | 0.3 | 64% |
| 30 | 0.2 | 100% |
| 31 | 54% | 88% |
| 32 | 0.6 | 100% |
| 33 | 0.2 | 95% |
| 34 | 0.4 | 84% |
| 35 | 0.06 | 100% |
| 36 | 0.5 | 56% |
| 37 | 0.5 | 95% |
| 38 | 0.9 | 93% |
| 39 | 0.2 | 94% |
| 40 | 0.2 | 100% |
| 41 | 0.5 | 100% |
| 42 | 0.4 | 61% |
| 43 | 1.6 | 100% |
| 44 | 2 | 69% |
| 45 | 1.8 | 100% |
| 46 | 1 | 64% |
| 47 | 1 | 100% |
| 48 | 1.5 | 1.5 μM |
| 49 | 1.2 | 100% |
| 50 | 0.5 | 100% |
| 51 | 1.2 | 90% |
| 52 | 0.4 | 81% |
| 53 | 0.4 | 92% |
| 54 | 1.5 | 96% |
| 55 | 3.2 | 72% |
| 56 | 0.8 | 48% |
| 57 | 3 | 100% |
| 58 | 1 | 100% |
| 59 | 0.6 | 96% |
| 60 | 72% | 100% |
| 61 | 49% | 100% |
| 62 | 0.8 | 96% |
| 63 | 42% | 86% |
| 64 | 39% | 89% |
| 65 | 0.4 μM | 45% |

It is to be understood that the preceding description teaches the principles of the present invention, with examples thereof which have emphasized certain aspects. It will also be understood that the practice of the invention encompasses all of the usual variations, adaptations and modifications as come within the scope of the following claims and their equivalents. However, numerous other equivalents not specifically elaborated on or discussed may nevertheless fall within the spirit and scope of the invention and claims and are intended to be included.

All documents cited herein are incorporated by reference.

What is claimed is:

1. A compound of Formula (I): 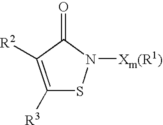
and pharmaceutically acceptable forms thereof, wherein:
X is —(CHR₄)—;
m is 0, 1, 2 or 3;
R¹ is -aryl-R₅, -heterocyclyl-R₆R₇ or —(C₃-C₁₄) cycloalkyl-R₆;
R² and R³ are each hydrogen or halogen;
R₄ is hydrogen, lower alkyl or R¹;
R₅ is hydrogen, halogen, heterocyclyl-R₇R_{8,} acyl, lower alkenyl-R_{9,} lower alkynyl-R_{9,} —SO₂—R_{10,} -carbonylaryl-R₉or alkoxycarbonyl-;
R⁶ is hydrogen, lower alkyl, acyl, halogen, aryl or heterocyclyl;
R₇ and R₈ are each hydrogen, lower alkyl or halogen;
R₉ is hydrogen, acyl or aryl, wherein aryl is optionally substituted by one or more nitrile or halogen;
R₁₀ is alkyl or aryl, wherein aryl is optionally substituted by one or more halogen;
wherein when XR¹ is —(CH₂)ₘ-(heterocyclyl)-R₆R₇, then heterocyclyl is other than pyrrolidinyl, 3(2H)-isothiazolone, piperidinyl, morpholinyl, benzisothiazol-3(2H)-one and 4H-1,3,2-benzoxazaphosphorin-4-one.

2. The compound of claim 1 wherein X is —(CHR₄)—.

3. The compound of claim 1 wherein m is 0 or 1.

4. The compound of claim 1 wherein R¹ is -aryl-R₅, -heterocyclyl-R₆R₇ or —(C₃-C₁₄) cycloalkyl-R₆ and heterocyclyl is other than pyrrolidinyl, 3(2H)-isothiazolone, piperidinyl, morpholinyl, benzisothiazol-3(2H)-one and 4H-1,3,2-benzoxazaphosphorin-4-one.

5. The compound of claim 1 wherein R² and R³ are each hydrogen or halogen.

6. The compound of claim 1 wherein R₄ is hydrogen, lower alkyl or R¹.

7. The compound of claim 1 wherein R₅ is hydrogen, heterocyclyl-R₇R₈, acyl, lower alkenyl-R₉, lower alkynyl-R₉, —SO₂-R₁₀, -carbonylaryl-R₉ or alkoxycarbonyl-.

8. The compound of claim 1 wherein R₆ is hydrogen, lower alkyl, halogen, aryl or heterocyclyl.

9. The compound of claim 1 wherein R₇ and R₈ are each hydrogen, lower alkyl or halogen.

10. The compound of claim 1 wherein R₉ is hydrogen, acyl or aryl, wherein aryl is optionally substituted by one or more nitrile or halogen.

11. The compound of claim 1 wherein Rio is alkyl or aryl, wherein aryl is optionally substituted by one or more halogen.

12. A compound and a pharmaceutically acceptable form thereof selected from the group consisting of

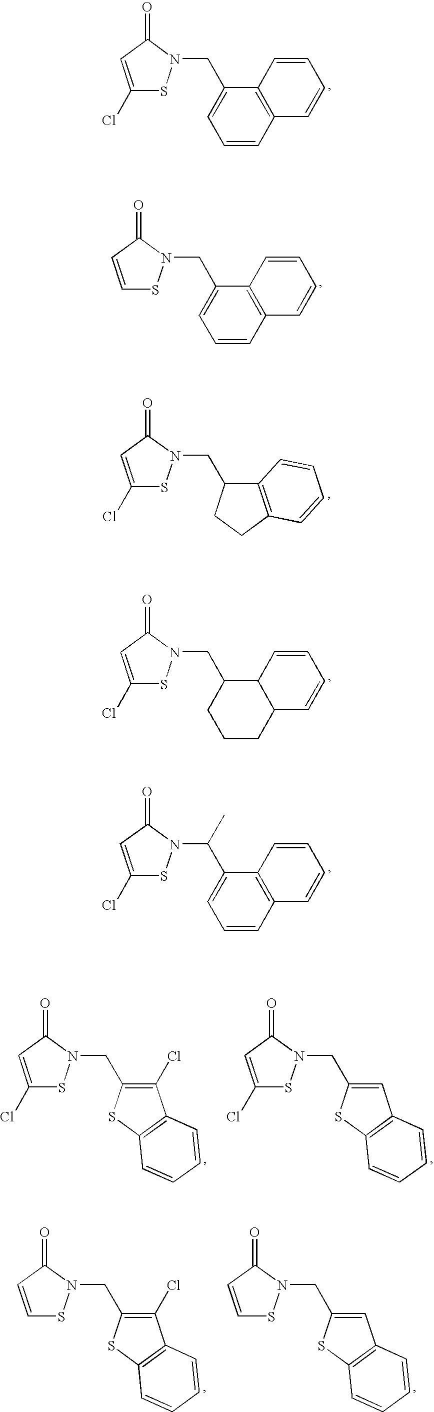

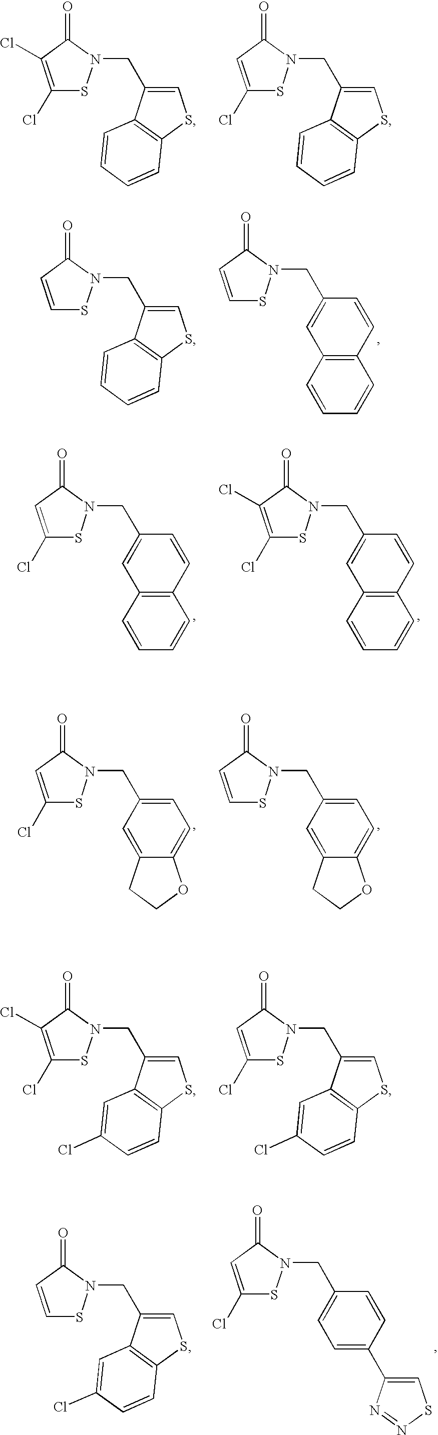

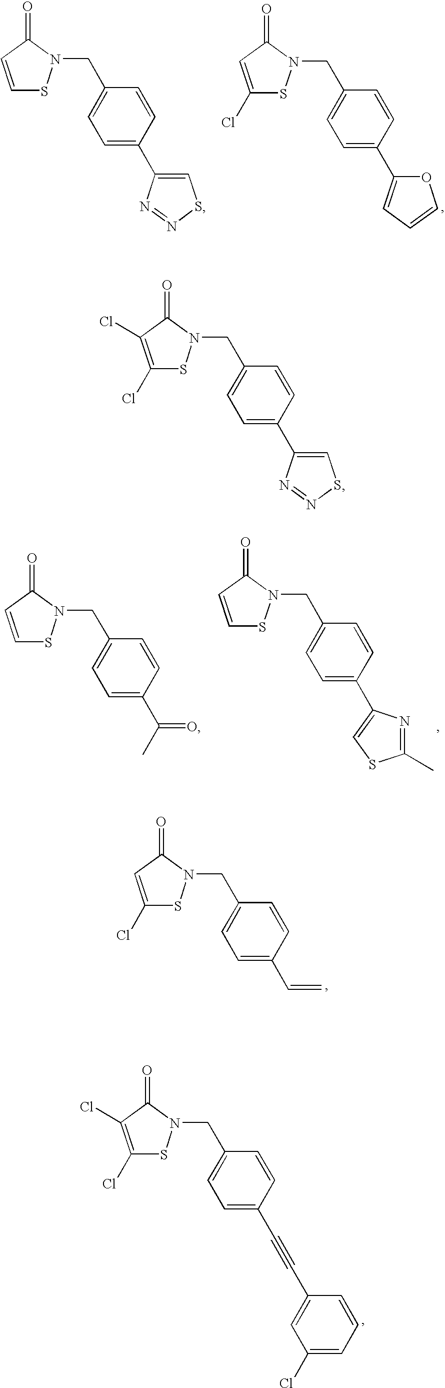

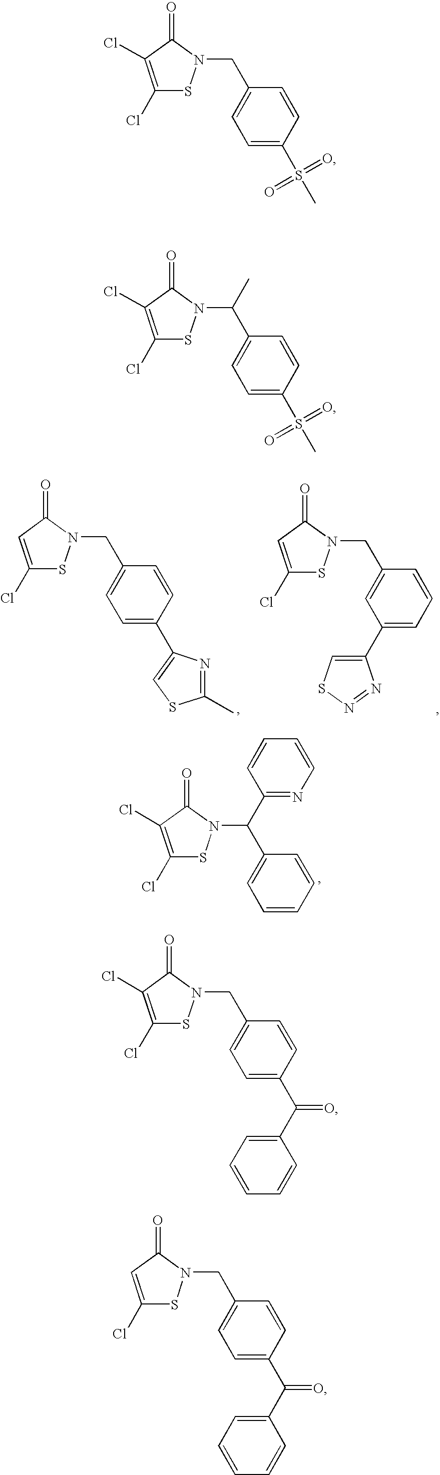

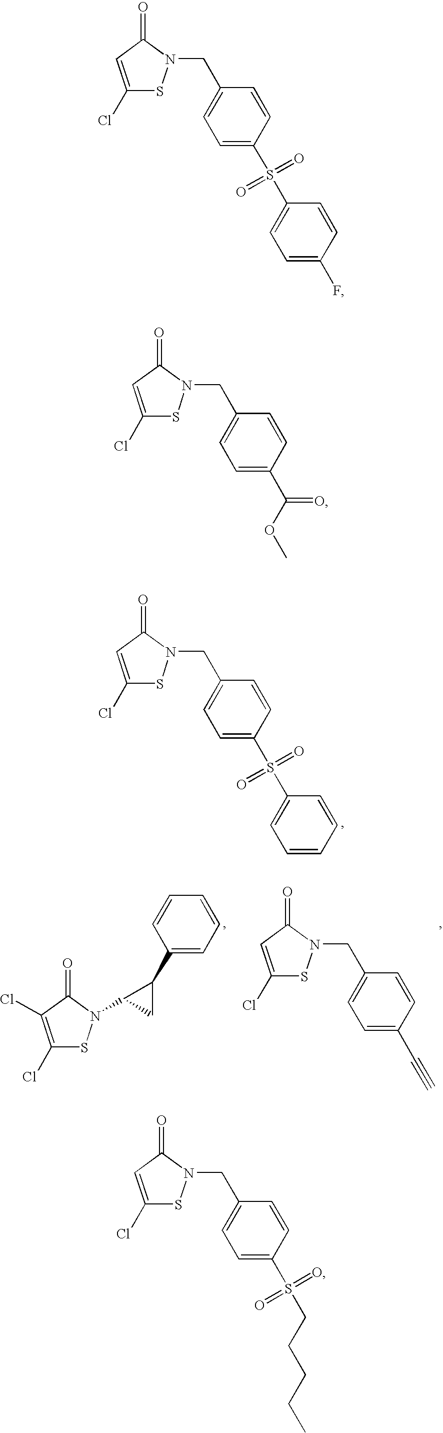

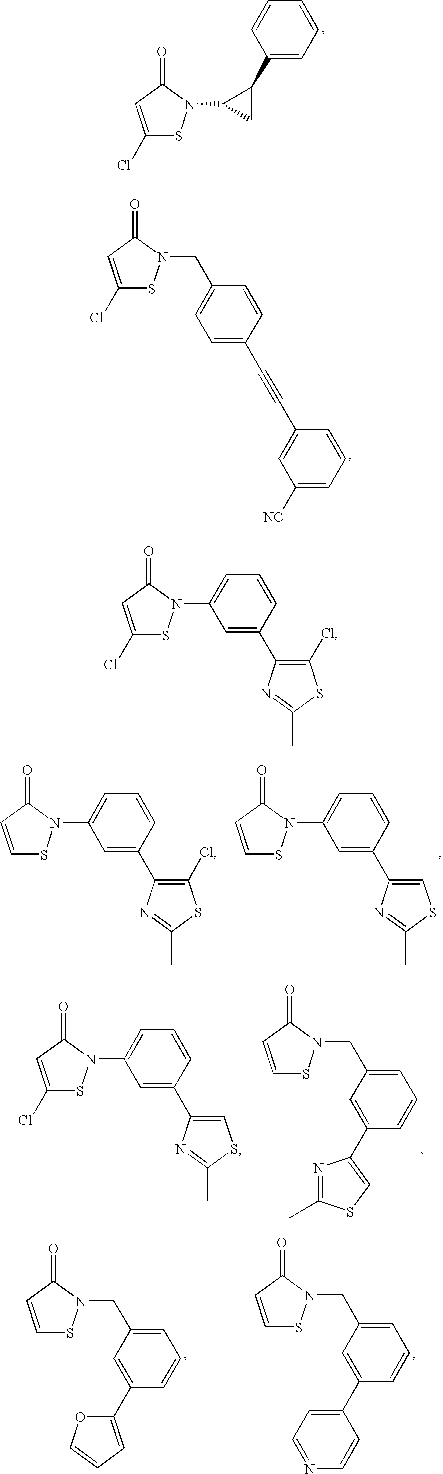

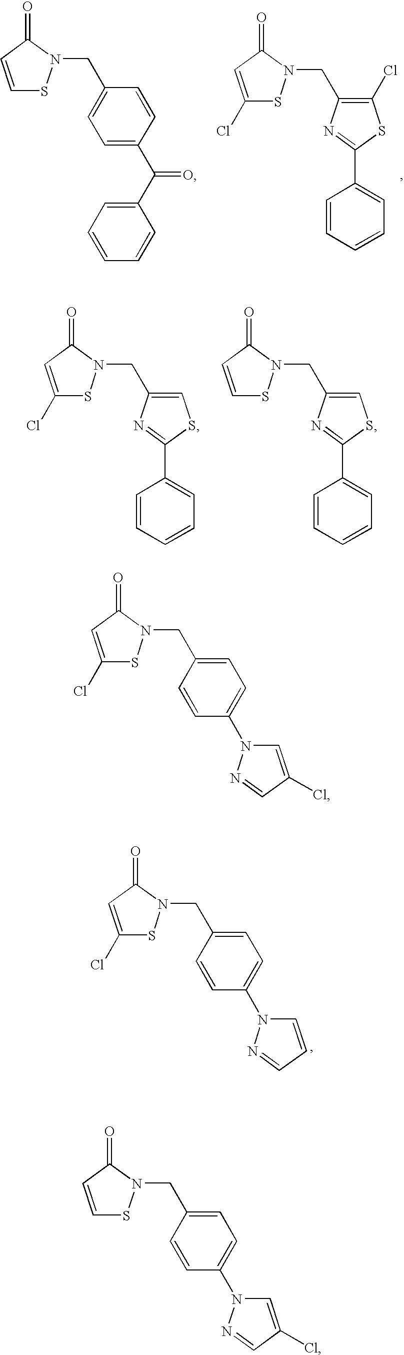

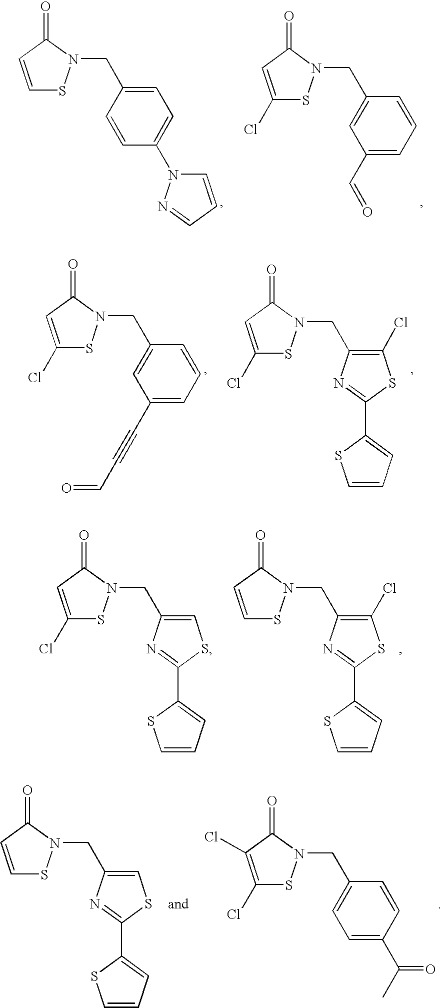

13. A pharmaceutical composition comprising the compound of claim 1 and a pharmaceutically acceptable carrier.

14. A pharmaceutical composition made by mixing a compound of claim 1 and a pharmaceutically acceptable carrier.

15. A process for making a pharmaceutical composition comprising mixing a compound of claim 1 and a pharmaceutically acceptable carrier.

16. A method for preventing, treating or ameliorating a cPLA₂ mediated inflammatory related disease, disorder or condition in a patient in need thereof comprising administering to the patient an effective amount of a compound of claim 1.

17. The method of claim 16, wherein the effective amount is in a range from about 0.001 mg to about 300 mg/kg of body weight per day.

18. The method of claim 16, wherein the disease, disorder or condition is selected from rheumatoid arthritis, inflammatory bowel disease, septic shock, osteoporosis, osteoarthritis, neuropathic pain, psoriasis, allergic inflammation in the lung, chronic obstructive pulmonary disorders, multiple sclerosis, Alzheimer's disease, stroke, ischemia or schizophrenia.

19. The method of claim 16, wherein the cPLA₂ mediated inflammatory related disease, disorder or condition results from the cellular secretion of TXB₂ or LTB₄.
